(19) United States
(12) Patent Application Publication
(10) Pub. No.: US 2026/0199421 A1
(43) Pub. Date: Jul. 16, 2026

(54) DOG FOOD ADDITIVE

(71) Applicant: THAROS LIMITED, Aldridge, Walsall (GB)

(72) Inventors: Rosemary Hope WARING, Aldridge (GB); Benjamin Eliezer NEDAS, Aldridge (GB)

(73) Assignee: THAROS LIMITED, Aldridge, Walsall (GB)

(21) Appl. No.: 19/135,671

(22) PCT Filed: Dec. 5, 2023
(86) PCT No.: PCT/GB2023/053136

§ 371 Date: Jun. 4, 2025

(30) Foreign Application Priority Data
Dec. 5, 2022 (GB) 2218268.7

(51) Int. Cl. A61K 36/8998; A23K 10/30; A23K 20/105; A23K 20/111; A23K 20/158; A23K 20/163; A23K 20/189; A23K 50/40; A61K 38/47; A61K 38/50; A61P 1/00; A61P 1/12; A61P 31/04

(52) U.S. Cl. CPC A61K 36/8998; A23K 10/30; A23K 20/105; A23K 20/111; A23K 20/158; A23K 20/163; A23K 20/189; A23K 50/40; A61K 38/47; A61K 38/50; A61P 1/00; A61P 1/12; A61P 31/04; C12Y 302/01001; C12Y 302/01008; C12Y 302/0108; C12Y 305/01

(57) ABSTRACT

The invention relates to a feed additive for use in increasing the diversity of gut bacteria in a dog is provided, the feed additive comprising a malt extract, the malt extract comprising one or more enzymes selected from the group consisting of amylases, maltases, cellulases, fructanases, glucanases, xylanases and deacetylases.

The invention relates to a feed additive for use in increasing the diversity of gut bacteria in a dog is provided, the feed additive comprising a malt extract, the malt extract comprising one or more enzymes selected from the group consisting of amylases, maltases, cellulases, fructanases, glucanases, xylanases and deacetylases.

Dogs originally diverged from wolves about 12,000 years ago when some animals began to accompany early hunters. They diverged further as their diet began to include starch when early human populations introduced agriculture into their lifestyles. Starch is made up of carbohydrate polymers with glucose subunits and is metabolised in the gastrointestinal tract by the enzyme amylase, which is released from the salivary gland and the pancreas (alpha-amylase) and the small intestine (gamma-amylase), into maltose (di-glucose) and then glucose which is absorbed by the bloodstream to provide energy to the body.

In general, working dogs, which were kept with little starch in the diet, have lower pancreatic amylase levels and are genetically less efficient at digesting non-meat diets. Dogs kept as companion animals (usually bred within the last 200 years) are often more adapted to our diets although individual variation occurs. The ancestors of dogs were almost entirely carnivorous, however modern canines are often fed a diet containing large amounts of starch and some vegetable matter. This may be from commercial feedstuffs or when they share bread, toast, pizza crust, rice, etc., with their owners.

Unlike humans, according to Pasha et al. (‘The saliva proteome of dogs: variations within and between breeds and between species’, Proteomics, 18, 3-4 (2018)), dogs have low salivary amylase so dietary starch is not hydrolysed before reaching the pancreatic enzymes released into the gastrointestinal tract. Many dogs have relatively low levels of pancreatic alpha-amylase (AMY2), the main enzyme which hydrolyses dietary starch. This varies between breeds and between individuals with, according to Freedman et al. (‘Genome sequencing highlights the dynamic early history of dogs’, PLOS Genetics, 10, 1 (2014)), those more closely related to wolves having lower copy numbers of the AMY2 gene and so lower enzyme activity.

Low levels of carbohydrate are metabolised by amylase and then the microbiome release short chain fatty acids (SCFA) which are readily absorbed as metabolic substrates. However when the upper gastrointestinal tract is overloaded with carbohydrates, undigested starch reaches the lower bowel where it is fermented by the gut microbiome. This leads to an alteration in the dominant species of bacteria and these changes are linked with intestinal dysfunction such as, according Omori et al. (‘Faecal microbiome in dogs with inflammatory bowel disease and intestinal lymphoma’, J. Vet. Med. Sci., 79, 11, 1840 (2017)) and Minamoto et al. (‘Alteration of the faecal microbiota and serum metabolite profiles in dogs with idiopathic inflammatory bowel disease’, Gut Microbes, 6, 1, 33 (2015)) to canine inflammatory bowel disease (CIBD) and, according to Hullar et al. (‘The canine gut microbiome is associated with higher risk of gastric dilatation volvulus and high risk genetic variants of the immune system’, PLOS ONE, 13, 6 (2018)), gastric dilation-volvulus (GDV) in large and giant dog breeds.

Currently commercial dog food falls into two main groups. There is the niche specialist market where the diet is largely protein, however this is expensive and many dogs are instead fed a high-carbohydrate diet which can include both commercial preparations and carbohydrate scraps from their human owners, such as sandwich crusts and pieces of pizza. The results of a survey (September 2019) by the French pet food manufacturers Royal Canin (RTM) found that 35% of dog owners fed their pet crisps as a treat while about 33% of dog owners fed their pet pizza, sausage rolls and snack junk food, although this left the animals with digestive issues.

Low amylase levels mean that a high-carbohydrate diet is not readily digested and absorbed early in its passage through the gut. More material is available to be fermented in the lower bowel and this modifies the dog's faecal microbiome to a more toxic profile which can have serious consequences for canine health. These include chronic inflammation and greater gut permeability, leading to allergies.

Canine allergies are becoming more common and have been identified by vets as an increasingly serious problem. Environmental factors such as diet are thought to be involved.

According to Olivry et al. (‘Critically appraised topic on AFRs of companion animals (7): signalment and cutaneous manifestations of dogs and cats with adverse food reactions’, BMC Vet. Res., 15, 1, 140 (2019)), of dogs with canine adverse food reactions, 22% were under 6 months when symptoms first appeared. Recently, it has become clear that the gut microbiome is a major component in regulation of the immune system response. According to Tan et al. (‘Dietary fibre and bacterial SCFA enhance oral tolerance and protect against food allergy through diverse cellular pathways’, Cell Reports, 15, 12, 2809 (2016)), dysbiosis of gut microbiota has been implicated in the development of allergy and, according to Mckenzie et al. (‘The nutrition-gut microbiome-physiology axis and allergic disease’, Immunological Reviews, 278, 1, 277 (2017)), short chain fatty acids modulate inflammatory responses via G-protein-coupled receptors and that high levels of SCFAs are protective against food allergies.

Clearly, a canine food supplement which could optimise the existing microbiome would potentially be useful in treating canine chronic enteropathies (CCEs) and allergies.

### SUMMARY OF THE INVENTION

In a first aspect of the invention, a feed additive for use in increasing the diversity of gut bacteria in a dog is provided, the feed additive comprising a malt extract, the malt extract comprising one or more enzymes selected from the group consisting of amylases, maltases, cellulases, fructanases, glucanases, xylanases and deacetylases.

In a second aspect of the invention, a feed additive for use in reducing diarrhoea in a dog is provided, the feed additive comprising a malt extract, the malt extract comprising one or more enzymes selected from the group consisting of amylases, maltases, cellulases, fructanases, glucanases, xylanases and deacetylases, preferably wherein the dog has a weight of at least 20, 21, 22, 23, 24, 25 kg, preferably a weight of 20-200, 20-175, 20-150, 20-125, 20-100, 20-50 kg.

In a third aspect of the invention, a feed additive for use in decreasing the level of a harmful digestive metabolite or increasing the level of a beneficial digestive metabolite in a dog gut is provided, the feed additive comprising a malt extract, the malt extract comprising one or more enzymes selected from the group consisting of amylases, maltases, cellulases, fructanases, glucanases, xylanases and deacetylases,
wherein the harmful digestive metabolite is selected from the group consisting of ammonia, methanol, ethanol, hydrogen sulphide, toluene, butanol, methanethiol, and dimethylsulfide, and
wherein the beneficial digestive metabolite is selected from the group consisting of acetone, acetic acid, butyric acid, and propionic acid.

In a fourth aspect of the invention, a feed additive for use in increasing the food appetite of a dog is provided, the feed additive comprising a malt extract, the malt extract comprising one or more enzymes selected from the group consisting of amylases, maltases, cellulases, fructanases, glucanases, xylanases and deacetylases.

In a fifth aspect of the invention, a feed additive for use in increasing the amount of bacterial genus Faecalbacterium in a dog digestive system is provided, the feed additive comprising a malt extract, the malt extract comprising one or more enzymes selected from the group consisting of amylases, maltases, cellulases, fructanases, glucanases, xylanases and deacetylases.

In a sixth aspect of the invention, a feed additive for use in decreasing the amount of bacterial genera Streptococcus and/or Treponema in a dog digestive system is provided, the feed additive comprising a malt extract, the malt extract comprising one or more enzymes selected from the group consisting of amylases, maltases, cellulases, fructanases, glucanases, xylanases and deacetylases.

In a seventh aspect of the invention, feed additive for use in preventing or treating Lyme disease in a dog is provided, the feed additive comprising a malt extract, the malt extract comprising one or more enzymes selected from the group consisting of amylases, maltases, cellulases, fructanases, glucanases, xylanases and deacetylases.

In an eighth aspect of the invention, a feed additive for use in reducing or preventing the spread of Lyme disease by dogs is provided, the feed additive comprising a malt extract, the malt extract comprising one or more enzymes selected from the group consisting of amylases, maltases, cellulases, fructanases, glucanases, xylanases and deacetylases.

Further aspects of the invention include a method for increasing the diversity of gut bacteria in a dog in need therefor, a method of preventing or treating diarrhoea in a dog in need therefor, preferably wherein the dog has a weight of at least 20, 21, 22, 23, 24, 25 kg, preferably a weight of 20-200, 20-175, 20-150, 20-125, 20-100, 20-50 kg, a method for decreasing the level of a harmful digestive metabolite or increasing the level of a beneficial digestive metabolite in the gut of a dog in need therefor, a method for increasing the food appetite of a dog, a method for increasing the amount of bacterial genus Faecalbacterium in a dog digestive system, a method for decreasing the amount of bacterial genera Streptococcus and/or Treponema in a dog digestive system, a method for preventing or treating Lyme disease in a dog, or a method for reducing or preventing the spread of Lyme disease by dogs, the method comprising the step of administering a therapeutic amount of a feed additive, the feed additive comprising a malt extract, the malt extract comprising one or more enzymes selected from the group consisting of amylases, maltases, cellulases, fructanases, glucanases, xylanases and deacetylases.

Further aspects of the invention include use of a feed additive for the manufacture of a medicament for increasing the diversity of gut bacteria in a dog, for reducing diarrhoea in a dog, preferably wherein the dog has a weight of at least 20, 21, 22, 23, 24, 25 kg, preferably a weight of 20-200, 20-175, 20-150, 20-125, 20-100, 20-50 kg, for decreasing the level of a harmful digestive metabolite or increasing the level of a beneficial digestive metabolite in a dog gut, for increasing the food appetite of a dog, for increasing the amount of bacterial genus Faecalbacterium in a dog digestive system, for decreasing the amount of bacterial genera Streptococcus and/or Treponema in a dog digestive system, for preventing or treating Lyme disease in a dog, or for reducing or preventing the spread of Lyme disease by dogs, wherein in all aspects the feed additive comprises a malt extract, the malt extract comprising one or more enzymes selected from the group consisting of amylases, maltases, cellulases, fructanases, glucanases, xylanases and deacetylases.

Further aspects of the invention include use of a feed additive for increasing the diversity of gut bacteria in a dog, for reducing diarrhoea in a dog, preferably wherein the dog has a weight of at least 20, 21, 22, 23, 24, 25 kg, preferably a weight of 20-200, 20-175, 20-150, 20-125, 20-100, 20-50 kg, for decreasing the level of a harmful digestive metabolite or increasing the level of a beneficial digestive metabolite in a dog gut, for increasing the food appetite of a dog, for increasing the amount of bacterial genus Faecalbacterium in a dog digestive system, for decreasing the amount of bacterial genera Streptococcus and/or Treponema in a dog digestive system, for preventing or treating Lyme disease in a dog, or for reducing or preventing the spread of Lyme disease by dogs, wherein in all aspects the feed additive comprises a malt extract, the malt extract comprising one or more enzymes selected from the group consisting of amylases, maltases, cellulases, fructanases, glucanases, xylanases and deacetylases.

### BRIEF DESCRIPTION OF THE FIGURES

The invention is described in more detail with reference to:

FIG. 1 which is a graph of count of genus versus dog before and after administration of EquiNectar™;

FIG. 2 which shows a list of bacterial FIG. 2 shows a list of bacterial genera and faecal volatile metabolites (VOCs) associated with ‘good’ and ‘bad’ health;

FIG. 3 which shows the top 25 abundant bacterial genera changes per dog (all not on antibiotics) after administration of Equinectar™;

FIGS. 4a and 4b which show scatter plots comparing pre- (x axis) and post-(y axis) administration of Equinectar™ for ‘good’ bacterial genera relative abundance (via percent hits) per dog (all not on antibiotics) and only those aged at least 5 years (not on antibiotics) respectively;

FIGS. 5a and 5b which show scatter plots comparing pre- (x axis) and post-(y axis) administration of Equinectar™ for ‘bad’ bacterial genera relative abundance (via percent hits) per dog (all not on antibiotics) and only those aged at least 5 years (not on antibiotics) respectively;

FIG. 6 which shows the volatile organic compound (VOC) changes per dog (all not on antibiotics) after administration of Equinectar™;

FIG. 7 which shows a scatter plot comparing pre- (x axis) and post-(y axis) administration of Equinectar™ for ‘bad’ VOCs (VOCs related to poor health) relative abundance (via percent hits) per dog (all not on antibiotics);

FIG. 8 which shows ‘bad’ VOCs versus ‘bad’ bacterial genera per dog (all dogs not on antibiotics) pre- and post-administration of Equinectar™; and

FIG. 9 which shows changes in the appetite, behaviour, body, coat, droppings and focus of the dogs (not on antibiotics).

### DETAILED DESCRIPTION OF THE INVENTION

In a first aspect of the invention, a feed additive for use in increasing the diversity of gut bacteria in a dog is provided, the feed additive comprising a malt extract, the malt extract comprising one or more enzymes selected from the group consisting of amylases, maltases, cellulases, fructanases, glucanases, xylanases and deacetylases.

In a second aspect of the invention, a feed additive for use in reducing diarrhoea in a dog is provided, the feed additive comprising a malt extract, the malt extract comprising one or more enzymes selected from the group consisting of amylases, maltases, cellulases, fructanases, glucanases, xylanases and deacetylases, preferably wherein the dog has a weight of at least 20, 21, 22, 23, 24, 25 kg, preferably a weight of 20-200, 20-175, 20-150, 20-125, 20-100, 20-50 kg.

In a third aspect of the invention, a feed additive for use in decreasing the level of a harmful digestive metabolite or increasing the level of a beneficial digestive metabolite in a dog gut is provided, the feed additive comprising a malt extract, the malt extract comprising one or more enzymes selected from the group consisting of amylases, maltases, cellulases, fructanases, glucanases, xylanases and deacetylases,
wherein the harmful digestive metabolite is selected from the group consisting of ammonia, methanol, ethanol, hydrogen sulphide, toluene, butanol, methanethiol, and dimethylsulfide, and
wherein the beneficial digestive metabolite is selected from the group consisting of acetone, acetic acid, butyric acid, and propionic acid.

In a fourth aspect of the invention, a feed additive for use in increasing the food appetite of a dog is provided, the feed additive comprising a malt extract, the malt extract comprising one or more enzymes selected from the group consisting of amylases, maltases, cellulases, fructanases, glucanases, xylanases and deacetylases.

In a fifth aspect of the invention, a feed additive for use in increasing the amount of bacterial genus Faecalbacterium in a dog digestive system is provided, the feed additive comprising a malt extract, the malt extract comprising one or more enzymes selected from the group consisting of amylases, maltases, cellulases, fructanases, glucanases, xylanases and deacetylases.

In a sixth aspect of the invention, a feed additive for use in decreasing the amount of bacterial genera Streptococcus and/or Treponema in a dog digestive system is provided, the feed additive comprising a malt extract, the malt extract comprising one or more enzymes selected from the group consisting of amylases, maltases, cellulases, fructanases, glucanases, xylanases and deacetylases.

In a seventh aspect of the invention, feed additive for use in preventing or treating Lyme disease in a dog is provided, the feed additive comprising a malt extract, the malt extract comprising one or more enzymes selected from the group consisting of amylases, maltases, cellulases, fructanases, glucanases, xylanases and deacetylases.

In an eighth aspect of the invention, a feed additive for use in reducing or preventing the spread of Lyme disease by dogs is provided, the feed additive comprising a malt extract, the malt extract comprising one or more enzymes selected from the group consisting of amylases, maltases, cellulases, fructanases, glucanases, xylanases and deacetylases.

Other aspects of the inventions are set forth in the Summary of the Invention hereinabove.

The malt extract may additionally comprise one or more proteinases and/or lipases.

The malt extract may additionally comprise one or more water soluble sugars selected from the group consisting of maltose, maltotriose, and maltose polymers. Preferably, the diastatic power of the malt extract is above 35, 40, 45, 50, 55, 60, 65, 70, 75 or 80 degrees Lintner.

The malt extract may be based on one of the seeds selected from the group consisting of barley, wheat, triticale, sorghum, maize, buck wheat, rice and a mixture thereof.

Typically, the daily dosage of the malt extract is 0.3-30, 0.4-20, 0.5-10, 0.7-5, 1-3 g per 1000 g dog body weight.

The feed additive may additionally comprises one or more medium chain triglycerides.

Typically the feed additive comprises 1-5, 1-10 or 1-15% w/w one or more medium chain triglycerides. Preferably, the medium chain triglyceride is coconut oil.

The feed additive may additionally comprises an effective amount of one or more water soluble organic acid or salt thereof. Typically, the water soluble organic acid is selected from the group consisting of caprylic acid, sorbic acid, caproic acid, benzoic acid, ascorbic acid, propionic acid, acetic acid, formic acid, fumaric acid, and tartaric acid. The feed additive may comprises 0.001-5, 0.01-3, 0.05-2, 0.1-1.5, 0.5-1.25, 0.75-1.2% w/w of one or more water soluble organic acid or salt thereof.

The feed additive for use according to any one of the preceding aspects of the invention may be used in combination with dog feed.

The barley malt extract comprises a plurality of enzymatically active digestive enzymes, in particular alpha- and/or beta-amylase (alpha-amylase breaks starch down yielding maltotriose and maltose from amylose, and maltose, glucose and limit dextrin from amylopectin and beta-amylase breaks down starch into maltose), maltase, cellulase, fructanases (which break down fructans found in grass), glucanases (which break down glucans found in cell walls), xylanases (which break down xylans in plant cell walls), deacetylases (which cleave acetyl groups from xylans and fructans thereby allowing xylanases and fructanases to break down the remainder of the molecular structure), and smaller amounts of proteinases and lipases (which break down respectively proteins and fats). The malt extract also comprises maltose, maltotriose and maltose polymers, and, depending on the precise parameters used in the process of preparing the malt extract, peptides and/or amino acids. The malt extract does not, however, comprise starch because the starch in the barley seeds is broken down to produce a mixture of the aforementioned maltose, maltotriose and maltose polymers.

The barley malt extract is prepared by soaking barley seeds in water in order to germinate the seeds. Germination causes the seeds to produce a variety of enzymes that break down, for example, starches into sugars through the production of amylases and other carbohydrases, such as fructanases. The germination process also induces other enzymes such as proteases that break down proteins in the grain. Germination is halted by drying with hot air at a temperature of no higher than about 75, 70, 65, 60, 55, 50, 45 or 40 degrees centigrade thereby to produce a malt. Whilst higher temperatures may be used to dry the germinated seeds, such higher temperatures denature an ever greater proportion of the enzymes present in the malt.

The dried sprouted seeds are then milled and water is added and heated to at least about 40, 45, 50, 55, 60, 65 but below 75 or 70 degrees centigrade in order to form a mash, and stirred for about one hour. The enzymes present are active up to different temperatures. Thus proteases and beta-amylases are active up to about 50 degrees centigrade. Thereafter up to about 65 degrees centigrade alpha-amylases still degrade starch to sugars. The next step is separation of the residual solids (‘spent grain’) from the liquid (‘wort’). The wort is then concentrated by vacuum evaporation to provide an active enzyme rich malt extract, typically comprising about 80% w/w solids in a solution also rich in sugars.

The malt extract typically has a diastatic power value of above 35 degrees Lintner (94 degrees Windisch Kolbach (WK) units), or above 40, 45, 50, 55, 60, 65, 70, 75 or 80 degrees Lintner. For comparative purposes, a malt with enough power to self-convert starch to sugars has a diastatic power of about 35 degrees Lintner.

EquiNectar™ (Tharos Limited, London, England) is an animal feed additive comprising about 85% w/w an enzyme rich malt extract (ERME™) derived from barley, about 2% w/w coconut oil (a medium chain triglyceride (MCT) comprising medium chain fatty acids (C₆-C₁₂) (MCFAs)), the preservative potassium sorbate (sorbic acid is found in many plants including rowan berries), and water. ERME™ is an enzyme rich malt extract derived from barley comprising 15% w/w water.

Whilst EquiNectar™ comprises a malt extract based on barley seeds, potentially any seed may be used to produce the malt. For example, wheat, triticale, sorghum, maize, buck wheat or rice may be used.

According to page 3 (lines 8-12 and 30-32) of WO 2018/096334 (Pepsis Limited), the medium chain triglyceride may be useful for improving the digestion of food as well as increasing glycogen availability in muscles and providing additional energy. As pancreatic alpha-amylases are activated by fatty acids, the addition of MCT (which hydrolyses to fatty acids) boosts the activity of the enzymes in the gut. Attia et al. (‘The effects of different oil sources on performance, digestive enzymes, carcass traits, biochemical, immunological, antioxidant and morphometric responses of broiler chicks’, Frontiers in Veterinary Science, 7, 181 (2020)) describes a study showing that coconut oil increases gut amylase activity as well as providing significant increases in blood plasma antibodies immunoglobulin G (IgG) and immunoglobulin M (IgM). It also improves antioxidant status, the antibody titre to avian influenza and the respiratory disease Newcastle disease, and the feed conversion ratio.

Whilst EquiNectar™ comprises coconut oil, other MCTs may be used including those having two or three different medium chain fatty acids (MCFAs) selected from the group consisting of caproic acid (C6), caprylic acid (C8), capric acid (C10) and lauric acid (C12).

Typically the animal feed additive preferably comprises about 1 to 5, 1 to 10, 1 to 15% w/w one or more MCTs. MCTs comprising at least 40 or 45% w/w lauric fatty acid are preferred, such as coconut oil, for the reasons provided hereinabove. Furthermore it has been observed by Hafeez et al. (‘Effect of diet supplemented with coconut essential oil on performance and villus histomorphology in broilers exposed to avian coccidiosis’, Tropical Animal Health and Production, 52, 5, 2499 (2020)) that using 2% coconut oil as a dietary supplement for broiler chicks led to significantly better feed conversion ratios than controls even when challenged with avian coccidiosis. The supplemented chicks also had improved gastrointestinal tract villus histology with higher length, width and surface area. In addition and as reported by Sefi et al. (‘Short chain fatty acids may improve hepatic mitochondrial energy efficiency in heat-stressed broilers’, J. Thermal Biology, 89, 102520 (2020)), as compared with other oils (long-chain saturated fatty acids in the form of beef tallow, monounsaturated fatty acids in the form of olive oil, and polyunsaturated fatty acids in the form of soybean oil), dietary coconut oil improves hepatic mitochondrial energy efficiency in heat-stressed broilers giving the highest adenosine triphosphate (ATP) concentration and mitochondrial membrane potential.

The feed additive preferably additionally comprises an effective amount of one or more water soluble organic acid or salt thereof. The water soluble organic acid is optionally selected from the group consisting of caprylic acid, sorbic acid, caproic acid, benzoic acid, ascorbic acid, propionic acid, acetic acid, formic acid, fumaric acid, and tartaric acid, and is preferably sorbic acid, more preferably a salt of sorbic acid, for example potassium sorbate. EquiNectar comprises about 1% w/w potassium sorbate. Thus the feed additive preferably comprises 0.001 to 5, 0.01 to 3, 0.05 to 2, 0.1 to 1.5, 0.5 to 1.25, 0.75 to 1.2% w/w of one or more water soluble organic acid or salt thereof.

### Example 1: Effect of EquiNectar™ on CCE's and Dog Allergies

Ten dogs of varying ages, breeds and weights were chosen to be a representative sample of companion animals and working dogs. Two were healthy, two had allergies and the rest had various forms of canine inflammatory bowel disease (CIBD).

The dogs were fed 15 ml EquiNectar™ twice daily for 2-3 weeks as a spread over their normal dry diet. EquiNectar™ (Tharos Limited, London, England) is an animal feed additive comprising an enzyme rich malt extract (ERME™) derived from barley, coconut oil (a medium chain triglyceride (MCT) comprising medium chain fatty acids (C₆-C₁₂) (MCFAs)), and the preservative potassium sorbate (sorbic acid is found in many plants including rowan berries). ERME™ is available from Muntons Limited (Stowmarket, UK) and is an enzyme-rich malt extract prepared by malting barley at a low temperature to retain the enzyme activity. This activity consists chiefly of amylase to digest starch, with fructanases, glucanases, glucosidases, some lipase and proteinase activity to break down all the constituents of plant cell walls and enable complete digestion of dietary carbohydrates.

The owners were asked to comment on whether feeding EquiNectar™ for two weeks altered the dog's general health or the microbiome as assessed by changes in faecal appearance and consistency. The results are summarised in Tables 1.1 to 1.3.

TABLE 1.1
General details on dogs administered 15 ml EquiNectar ™ twice daily.

| | Dates of | | Weight before/after | |
|---|---|---|---|---|
| Dog | administration of | | administration of | Age |
| number | EquiNectar ™ | Breed | EquiNectar ™ (kg) | (years) |
| 1 | 13-27 Aug. 2020 | Labrador | 38/38 | 9 |
| 2 | 13-17 Aug. 2020 | Cocker Spaniel | 12/12 | 4 |
| 3 | 10-26 Aug. 2020 | Whippet | 13.4/12.9 | 5 |
| 4 | 6-26 Aug. 2020 | German Shepherd | 33/34 | 8 |
| 5 | 6-24 Aug. 2020 | German Shepherd | 34/34 | 7 |
| 6 | 6-24 Aug. 2020 | German Shepherd | 35/35 | 7 |
| 7 | 10 Aug. to 12 Sep. 2020 | Lurcher/Weimaraner | 40/41 | 6.5 |
| 8 | 10-12 Aug. 2020 | Patterdale | 10/10 | 3 |
| 9 | 3-21 Sep. 2020 2020 | Jack Russell terrier | 8/8 | 8 |
| 10 | 3-21 Sep. 2020 | Sheepdog | 10/11 | 2 |

TABLE 1.2
Owner observations of dogs administered 15 ml EquiNectar ™ twice daily.

| Dog | | Compliance | Appetite | Behaviour | Condition ^{a} |
|---|---|---|---|---|---|
| number | Diet | problems | changes | changes | changes |
| 1 | Dry | None | None | None | Coat |
| | | | | | glossier |
| 2 | Dry | Refused to eat | — | — | — |
| | | EquiNectar ™ | | | |
| 3 | Dry | Ate EquiNectar ™ | None | Possibly | None |
| | | on biscuit | | slightly | |
| | | | | subdued | |
| 4 | Dry | None | Appetite | Seemed | Coat |
| | | | improved | brighter | glossier |
| 5 | Dry | None | None | | |
| 6 | Dry | None | None | | |
| 7 | Dry + Meat + | Ate EquiNectar ™ | None | Slightly | Coat slightly |
| | IAMS ™ | when spread on | | improved | glossier |
| | | dry feed | | | |
| 8 | Dry + Meat + | Refused to eat | — | — | — |
| | complete feed | EquiNectar ™ | | | |
| 9 | Dry | None | None | None | Coat |
| | | | | | glossier |
| 10 | Dry | None | None | None | Coat |
| | | | | | glossier |

^{a} Combination of shininess of coat without dandruff.

TABLE 1.3
Owner observations of dogs administered 15 ml EquiNectar ™ twice daily.

| Dog | Faecal changes | Allergies (pre-existing) | Skin problems | Health problems |
|---|---|---|---|---|
| 1 | None | None | None | None |
| 2 | — | — | — | — |
| 3 | Oilier | None | None | Had gastroenteritis 4 days after trial |
| 4 | None | Gluten (rash eczema) | Yes (pre-existing) | None |
| 5 | Smellier and lighter in colour | None | None | Pre-existing Panas^{a} |
| 6 | Smellier and lighter in colour | None | None | None |
| 7 | Slightly drier | None | None | None |
| 8 | — | — | — | — |
| 9 | Harder and drier | Gut allergy | None | None |
| 10 | Harder and drier | None | None | None |

^{a}A syndrome of allergies and digestive problems.

ERME™ contains low levels of proteinases which would break down potential allergens which are often proteins. Human intolerance to diet has recently been linked to the presence of complex carbohydrates and these would again be broken down by the range of enzymes in ERME™. The two dogs in the trial described as having allergies had improved condition (glossier coat, less itching).

CIBD is a relatively common digestive fault thought to be due to changes in the gut microbiome and presenting with chronic diarrhoea. It was observed that six out of the ten dogs showed changes in the faeces with three producing harder and drier faeces instead of the previous semi-diarrhoea. None of the dogs produced faeces with increased liquidity.

Larger dogs (>25 kg) are more susceptible to gut and digestive problems, probably because they have a relatively more developed caecum and colon and a longer colonic transit time. Dogs responding well to EquiNectar™ had a mean weight of 26 kg (8-40 Kg) while non-responders had a mean weight of 19 Kg (10-35 Kg). It was observed that dogs that were larger, had diarrhoea or allergies showed most improvement on supplementation with EquiNectar™.

### Example 2: Effect of EquiNectar™ on the Canine Gut Microbiome

Eighteen adult dogs were fed 15 ml EquiNectar™ twice daily for six weeks as a spread over their normal dry diet and faecal samples taken before the start and after the study was complete. The faecal samples were processed and the gut microbiome content was determined by 16S metagenomic analysis (Illumina Incorporated (CA, US) platform) and the gut microbiome profiled using error-corrected 454 pyrosequencing data from the 16S rRNA amplicons. This gave identification of the phyla, class, order, family, genus and species with estimates of the relative frequencies (percentage of total hits) is provided below in Tables 2.1 to 2.18.

TABLE 2.1
Top eight phyla, class, order, family, genus and species with estimates of the relative frequencies
(percentage of total hits) for dog 1 (Sally) before and after administration of EquiNectar ™.

| Before EquiNectar ™ | | After EquiNectar ™ | |
|---|---|---|---|
| Phylum | Frequency (%) | Phylum | Frequency (%) |
| Bacteroidetes | 34.50 | Firmicutes | 49.62 |
| Firmicutes | 31.24 | Bacteroidetes | 24.06 |
| Fusobacteria | 15.25 | Fusobacteria | 14.86 |
| Proteobacteria | 4.52 | Proteobacteria | 4.71 |
| Spirochaetes | 3.14 | Unclassified at Phylum level | 2.64 |
| Unclassified at Phylum level | 3.13 | Spirochaetes | 1.68 |
| Actinobacteria | 2.65 | Verrucomicrobia | 0.80 |
| Verrucomicrobia | 2.10 | Actinobacteria | 0.39 |
| Class | Frequency (%) | Class | Frequency (%) |
| Bacteroidia | 28.96 | Clostridia | 37.76 |
| Clostridia | 25.25 | Bacteroidia | 20.48 |
| Fusobacteria | 15.25 | Fusobacteria | 14.86 |
| Unclassified at Class level | 5.33 | Erysipelotrichi | 9.34 |
| Sphingobacteriia | 3.84 | Unclassified at Class level | 4.27 |
| Erysipelotrichi | 3.74 | Gammaproteobacteria | 2.50 |
| Spirochaetes | 3.13 | Sphingobacteriia | 2.27 |
| Actinobacteria | 2.63 | Bacilli | 2.03 |
| Order | Frequency (%) | Order | Frequency (%) |
| Bacteroidales | 28.96 | Clostridiales | 36.63 |
| Clostridiales | 23.03 | Bacteroidales | 20.48 |
| Fusobacteriales | 15.25 | Fusobacteriales | 14.86 |
| Unclassified at Order level | 5.83 | Erysipelotrichales | 9.34 |
| Sphingobacteriales | 3.84 | Unclassified at Order level | 4.67 |
| Erysipelotrichales | 3.74 | Sphingobacteriales | 2.27 |
| Spirochaetales | 3.03 | Spirochaetales | 1.66 |
| Bifidobacteriales | 2.22 | Lactobacillales | 1.50 |
| Family | Frequency (%) | Family | Frequency (%) |
| Fusobacteriaceae | 15.19 | Fusobacteriaceae | 14.80 |
| Bacteroidaceae | 14.30 | Clostridiaceae | 13.41 |
| Unclassified at Family level | 8.72 | Bacteroidaceae | 11.34 |
| Lachnospiraceae | 8.42 | Peptostreptococcaceae | 8.75 |
| Prevotellaceae | 6.54 | Lachnospiraceae | 8.39 |
| Clostridiaceae | 5.83 | Unclassified at Family level | 7.20 |
| Paraprevotellaceae | 3.71 | Coprobacillaceae | 6.34 |
| Ruminococcaceae | 3.59 | Prevotellaceae | 4.49 |
| Genus | Frequency (%) | Genus | Frequency (%) |
| Bacteroides | 14.30 | Unclassified at Genus level | 12.33 |
| Unclassified at Genus level | 13.28 | Bacteroides | 11.34 |
| Fusobacterium | 11.79 | Fusobacterium | 10.59 |
| Prevotella | 9.71 | Alkaliphilus | 8.84 |
| Blautia | 5.54 | Tepidibacter | 7.04 |
| Clostridium | 4.83 | Prevotella | 6.24 |
| Treponema | 3.03 | Blautia | 5.90 |
| Ruminococcus | 2.25 | Catenibacterium | 5.22 |
| Species | Frequency (%) | Species | Frequency (%) |
| Unclassified at Species level | 39.98 | Unclassified at Species level | 44.68 |
| Fusobacterium gonidiaformans | 11.60 | Fusobacterium gonidiaformans | 10.37 |
| Prevotella copri | 5.61 | Alkaliphilus crotonatoxidans | 5.88 |
| Bacteroides denticanum | 5.10 | Bacteroides denticanum | 3.99 |
| Bacteroides rodentium | 2.33 | Prevotella copri | 3.83 |
| Bacteroides coprocola | 2.28 | Blautia coccoides | 3.23 |
| Blautia coccoides | 1.80 | Eubacterium biforme | 1.88 |

TABLE 2.2
Top eight phyla, class, order, family, genus and species with estimates
of the relative frequencies (percentage of total hits) for dog 2
(Reggie) before and after administration of EquiNectar ™.

| | |
|---|---|
| Before EquiNectar ™ | After EquiNectar ™ |

| Phylum | Frequency (%) | Phylum | Frequency (%) |
|---|---|---|---|
| Bacteroidetes | 34.68 | Firmicutes | 80.98 |
| Firmicutes | 31.59 | Bacteroidetes | 13.82 |
| Fusobacteria | 7.34 | Proteobacteria | 2.40 |
| Spirochaetes | 5.67 | Unclassified at Phylum | 1.75 |
| | | level | |
| Verrucomicrobia | 4.51 | Tenericutes | 0.31 |
| Unclassified at | 4.43 | Actinobacteria | 0.21 |
| Phylum level | | | |
| Proteobacteria | 4.29 | Fusobacteria | 0.15 |
| Fibrobacteres | 3.94 | Cyanobacteria | 0.10 |

| Class | Frequency (%) | Class | Frequency (%) |
|---|---|---|---|
| Clostridia | 27.25 | Clostridia | 55.56 |
| Bacteroidia | 23.71 | Erysipelotrichi | 23.04 |
| Unclassified at Class | 8.53 | Bacteroidia | 12.72 |
| level | | | |
| Sphingobacteriia | 7.80 | Unclassified at Class | 2.58 |
| | | level | |
| Fusobacteria | 7.34 | Bacilli | 2.04 |
| Spirochaetes | 5.67 | Betaproteobacteria | 0.95 |
| Methylacidiphilae | 3.98 | Flavobacteriia | 0.73 |
| Fibrobacteria | 3.94 | Alphaproteobacteria | 0.47 |
| Order | Frequency (%) | Order | Frequency (%) |
| Clostridiales | 25.78 | Clostridiales | 55.34 |
| Bacteroidales | 23.71 | Erysipelotrichales | 23.04 |
| Unclassified at Order | 9.16 | Bacteroidales | 12.72 |
| level | | | |
| Sphingobacteriales | 7.80 | Unclassified at Order | 2.81 |
| | | level | |
| Fusobacteriales | 7.34 | Turicibacterales | 1.08 |
| Spirochaetales | 5.48 | Burkholderiales | 0.93 |
| Methylacidiphilales | 3.98 | Flavobacteriales | 0.73 |
| Fibrobacterales | 3.94 | Lactobacillies | 0.65 |

| Family | Frequency (%) | Family | Frequency (%) |
|---|---|---|---|
| Unclassified at | 13.51 | Veillonellaceae | 22.73 |
| Family level | | | |
| Lachnospiraceae | 8.53 | Peptostreptococcaceae | 15.47 |
| Bacteroidaceae | 8.28 | Erysipelotrichaceae | 12.66 |
| Fusobacteriaceae | 7.33 | Clostridiaceae | 10.94 |
| Sphingobacteriaceae | 6.77 | Coprobacillaceae | 10.34 |
| Prevotellaceae | 5.78 | Prevotellaceae | 5.20 |
| Ruminococcaceae | 5.66 | Bacteroidaceae | 5.20 |
| Spirochaetaceae | 5.48 | Unclassified at Family | 4.88 |
| | | level | |
| Genus | Frequency (%) | Genus | Frequency (%) |
| Unclassified at | 18.73 | Phascolarctobacterium | 15.12 |
| Genus level | | | |
| Bacteroides | 8.28 | Eubacterium | 12.24 |
| Prevotella | 6.64 | Clostridium | 11.14 |
| Fusobacterium | 6.02 | Catenibacterium | 10.30 |
| Treponema | 5.48 | Alkaliphilus | 8.67 |
| Blautia | 4.90 | Prevotella | 7.15 |
| Clostridium | 4.16 | Megamonas | 7.02 |
| Candidatus | 3.98 | Unclassified at Genus | 6.62 |
| Methylacidiphilum | | level | |

| Species | Frequency (%) | Species | Frequency (%) |
|---|---|---|---|
| Unclassified at | 47.86 | Unclassified at Species | 51.53 |
| Species level | | level | |
| Fusobacterium | 5.91 | Phascolarctobacterium | 11.96 |
| gonidiaformans | | succinatutens | |
| Prevotella copri | 3.95 | Eubacterium biforme | 11.32 |
| Sphingobacterium | 3.49 | Prevotella copri | |
| bambusae | | | |
| Fibrobacter | 3.35 | Alkaliphilus | 4.73 |
| succinogenes | | peptidifermentans | |
| Bacteroides | 2.84 | Megamonas funiformis | 2.66 |
| denticanum | | | |
| Dysgonomonas | 2.54 | Bacteroides coprocola | 1.93 |
| wimpennyi | | | |

TABLE 2.3
Top eight phyla, class, order, family, genus and species with estimates
of the relative frequencies (percentage of total hits) for dog 3
(Presley) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 63.86 | Firmicutes | 55.84 |
| Fusobacteria | 29.18 | Fusobacteria | 28.23 |
| Unclassified at Phylum | 2.80 | Proteobacteria | 11.76 |
| level | | | |
| Proteobacteria | 2.08 | Unclassified at Phylum | 2.94 |
| | | level | |
| Bacteroidetes | 1.31 | Bacteroidetes | 0.63 |
| Actinobacteria | 0.30 | Actinobacteria | 0.25 |
| Cyanobacteria | 0.15 | Spirochaetes | 0.07 |
| Spirochaetes | 0.09 | Tenericutes | 0.05 |
| Class | Frequency (%) | Class | Frequency (%) |
| Clostridia | 52.29 | Clostridia | 41.33 |
| Fusobacteria | 29.18 | Fusobacteria | 28.23 |
| Bacilli | 10.87 | Bacilli | 13.10 |
| Unclassified at Class | 3.70 | Gammaproteobacteria | 4.51 |
| level | | | |
| Bacteroidia | 1.02 | Epsilonproteobacteria | 3.76 |
| Gammaproteobacteria | 0.79 | Unclassified at Class | 3.64 |
| | | level | |
| Erysipelotrichi | 0.53 | Betaproteobacteria | 2.92 |
| Actinobacteria | 0.28 | Erysipelotrichi | 1.11 |
| Order | Frequency (%) | Order | Frequency (%) |
| Clostridiales | 46.96 | Clostridiales | 32.98 |
| Fusobacteriales | 29.18 | Fusobacteriales | 28.23 |
| Lactobacillus | 10.25 | Lactobacillies | 9.89 |
| Coriobacteriales | 5.24 | Coriobacteriales | 8.25 |
| Unclassified at Order | 3.85 | Enterobacteriales | 4.13 |
| level | | | |
| Bacteroidales | 1.02 | Unclassified at Order | 3.97 |
| | | level | |
| Erysipelotrichales | 0.53 | Campylobacterales | 3.76 |
| Turicibacterales | 0.43 | Turicibacterales | 2.89 |
| Family | Frequency (%) | Family | Frequency (%) |
| Lachnospiraceae | 29.32 | Fusobacteriaceae | 28.16 |
| Fusobacteriaceae | 29.14 | Clostridiaceae | 16.28 |
| Clostridiaceae | 16.04 | Lachnospiraceae | 9.57 |
| Streptococcaceae | 9.79 | Streptococcaceae | 9.54 |
| Coriobacteriaceae | 5.24 | Coriobacteriaceae | 8.25 |
| Unclassified at Family | 5.20 | Unclassified at Family | 5.26 |
| level | | level | |
| Bacteroidaceae | 0.78 | Enterobacteriaceae | 4.13 |
| Turicibacteraceae | 0.43 | Campylobacteraceae | 3.68 |
| Genus | Frequency (%) | Genus | Frequency (%) |
| Fusobacterium | 26.46 | Fusobacterium | 24.76 |
| Blautia | 14.91 | Clostridium | 13.98 |
| Clostridium | 14.24 | Streptococcus | 9.53 |
| Ruminococcus | 10.13 | Collinsella | 7.83 |
| Streptococcus | 9.79 | Unclassified at Genus | 7.48 |
| | | level | |
| Unclassified at Genus | 6.50 | Blautia | 4.30 |
| level | | | |
| Collinsella | 5.01 | Campylobacter | 3.67 |
| Cetobacterium | 1.99 | Ruminococcus | 3.01 |
| Species | Frequency (%) | Species | Frequency (%) |
| Unclassified at Species | 35.01 | Unclassified at Species | 32.13 |
| level | | level | |
| Fusobacterium | 25.96 | Fusobacterium | 24.12 |
| gonidiaformans | | gonidiaformans | |
| Ruminococcus gnavus | 8.42 | Collinsella intestinalis | 7.70 |
| Collinsella intestinalis | 4.88 | Streptococcus bovis | 4.88 |
| Blautia coccoides | 4.80 | Ruminococcus gnavus | 2.59 |
| Streptococcus bovis | 3.55 | Cetobacterium ceti | 2.50 |
| Blautia producta | 2.97 | Clostridium | 2.09 |
| | | perfringens | |

TABLE 2.4
Top eight phyla, class, order, family, genus and species with estimates
of the relative frequencies (percentage of total hits) for dog
4 (Lily) before and after administration of EquiNectar ™.

| Before EquiNectar ™ | | After EquiNectar ™ | |
|---|---|---|---|
| Phylum | Frequency (%) | Phylum | Frequency (%) |
| Bacteroidetes | 46.48 | Bacteroidetes | 42.12 |
| Firmicutes | 23.78 | Fusobacteria | 19.15 |
| Fusobacteria | 22.26 | Proteobacteria | 18.47 |
| Proteobacteria | 5.19 | Firmicutes | 17.00 |
| Unclassified at Phylum | 1.57 | Unclassified at Phylum | 1.98 |
| level | | level | |
| Spirochaetes | 0.21 | Actinobacteria | 0.43 |
| Actinobacteria | 0.17 | Spirochaetes | 0.34 |
| Cyanobacteria | 0.09 | Tenericutes | 0.20 |
| Class | Frequency (%) | Class | Frequency (%) |
| Bacteroidia | 44.78 | Bacteroidia | 39.12 |
| Fusobacteria | 22.26 | Fusobacteria | 19.15 |
| Clostridia | 20.82 | Clostridia | 14.57 |
| Erysipelotrichi | 2.23 | Epsilonproteobacteria | 13.13 |
| Unclassified at Class | 2.18 | Unclassified at Class | 3.03 |
| level | | level | |
| Betaproteobacteria | 1.65 | Betaproteobacteria | 2.76 |
| Epsilonproteobacteria | 1.55 | Flavobacteriia | 2.26 |
| Gammaproteobacteria | 1.54 | Gammaproteobacteria | 1.73 |
| Order | Frequency (%) | Order | Frequency (%) |
| Bacteroidales | 44.78 | Bacteroidales | 39.12 |
| Fusobacteriales | 22.26 | Fusobacteriales | 19.15 |
| Clostridiales | 30.32 | Clostridiales | 14.21 |
| Unclassified at Order | 2.36 | Campylobacterales | 13.13 |
| level | | | |
| Erysipelotrichales | 2.26 | Unclassified at Order | 3.31 |
| | | level | |
| Burkholderiales | 1.63 | Burkholderiales | 2.72 |
| Campylobacterales | 1.55 | Flavobacteriales | 2.26 |
| Aeromonadales | 1.30 | Erysipelotrichales | 1.68 |
| Family | Frequency (%) | Family | Frequency (%) |
| Bacteroidaceae | 23.04 | Bacteroidaceae | 22.41 |
| Fusobacteriaceae | 22.20 | Fusobacteriaceae | 19.06 |
| Prevotellaceae | 12.45 | Helicobacteraceae | 12.45 |
| Paraprevotellaceae | 7.88 | Paraprevotellaceae | 9.82 |
| Veillonellaceae | 6.92 | Unclassified at Family | 5.18 |
| | | level | |
| Lachnospiraceae | 5.95 | Prevotellaceae | 4.93 |
| Clostridiaceae | 4.83 | Lachnospiraceae | 4.28 |
| Unclassified at Family | 3.60 | Ruminococcaceae | 3.74 |
| level | | | |
| Genus | Frequency (%) | Genus | Frequency (%) |
| Bacteroides | 23.04 | Bacteroides | 22.41 |
| Prevotella | 20.29 | Prevotella | 14.70 |
| Fusobacterium | 17.76 | Fusobacterium | 13.87 |
| Unclassified at Genus | 6.74 | Helicobacter | 12.45 |
| level | | | |
| Megamonas | 4.15 | Unclassified at Genus | 9.00 |
| | | level | |
| Clostridium | 4.12 | Faecalibacterium | 3.39 |
| Blautia | 3.66 | Blautia | 3.00 |
| Phascolarctobacterium | 2.52 | Clostridium | 2.92 |
| Species | Frequency (%) | Species | Frequency (%) |
| Unclassified at Species | 34.72 | Unclassified at | 36.72 |
| level | | Species level | |
| Fusobacterium | 17.54 | Fusobacterium | 13.52 |
| gonidiaformans | | gonidiaformans | |
| Prevotella copri | 11.62 | Bacteroides | 8.13 |
| | | denticanum | |
| Bacteroides | 10.82 | Bacteroides rodentium | 6.95 |
| denticanum | | | |
| Bacteroides rodentium | 4.18 | Helicobacter cinaedi | 6.94 |
| Clostridium hiranonis | 2.33 | Prevotella copri | 3.95 |
| Phascolarctobacterium | 1.67 | Sutterella stercoricanis | 1.83 |
| succinatutens | | | |
| Cetobacterium ceti | 1.36 | Bacteroides | 1.74 |
| | | xylanisolvens | |

TABLE 2.5
Top eight phyla, class, order, family, genus and species with estimates
of the relative frequencies (percentage of total hits) for dog
5 (Lexi) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 34.93 | Firmicutes | 37.25 |
| Fusobacteria | 27.00 | Bacteroidetes | 36.90 |
| Bacteroidetes | 18.90 | Fusobacteria | 15.46 |
| Proteobacteria | 9.91 | Proteobacteria | 7.42 |
| Unclassified at Phylum | 5.42 | Unclassified at Phylum | 1.98 |
| level | | level | |
| Verrucomicrobia | 1.05 | Actinobacteria | 0.26 |
| Actinobacteria | 0.70 | Cyanobacteria | 0.16 |
| Spirochaetes | 0.69 | Tenericutes | 0.15 |
| Class | Frequency (%) | Class | Frequency (%) |
| Fusobacteria | 27.00 | Bacteroidia | 35.61 |
| Erysipelotrichi | 20.22 | Clostridia | 25.53 |
| Bacteroidia | 17.23 | Fusobacteria | 15.46 |
| Clostridia | 10.16 | Erysipelotrichi | 10.22 |
| Unclassified at Class | 8.48 | Betaproteobacteria | 3.71 |
| level | | | |
| Bacilli | 4.30 | Unclassified at Class | 2.95 |
| Betaproteobacteria | 3.41 | Gammaproteobacteria | 1.57 |
| Gammaproteobacteria | 3.22 | Epsilonproteobacteria | 1.41 |
| Order | Frequency (%) | Order | Frequency (%) |
| Fusobacteriales | 27.00 | Bacteroidales | 35.61 |
| Erysipelotrichales | 20.22 | Clostridiales | 24.13 |
| Bacteroidales | 17.23 | Fusobacteriales | 15.46 |
| Clostridiales | 9.21 | Erysipelotrichales | 10.22 |
| Unclassified at Order | 8.80 | Burkholderiales | 3.66 |
| level | | | |
| Lactobacillies | 3.77 | Unclassified at Order | 3.24 |
| | | level | |
| Burkholderiales | 3.37 | Campylobacterales | 1.41 |
| Chromatiales | 1.64 | Coriobacteriales | 1.32 |
| Family | Frequency (%) | Family | Frequency (%) |
| Fusobacteriaceae | 26.91 | Prevotellaceae | 18.84 |
| Coprobacillaceae | 19.85 | Fusobacteriaceae | 15.39 |
| Unclassified at Family | 10.16 | Bacteroidaceae | 12.75 |
| level | | | |
| Prevotellaceae | 7.99 | Coprobacillaceae | 8.02 |
| Bacteroidaceae | 5.59 | Lachnospiraceae | 7.51 |
| Alcaligenaceae | 3.28 | Ruminococcaceae | 6.31 |
| Lactobacilluse | 3.17 | Unclassified at Family | 5.52 |
| | | level | |
| Paraprevotellaceae | 2.64 | Clostridiaceae | 5.02 |
| Genus | Frequency (%) | Genus | Frequency (%) |
| Fusobacterium | 23.05 | Prevotella | 20.57 |
| Catenibacterium | 19.27 | Bacteroides | 12.75 |
| Unclassified at Genus | 13.70 | Fusobacterium | 11.26 |
| level | | Unclassified at Genus | 8.88 |
| Prevotella | 8.80 | level | |
| Bacteroides | 5.59 | Catenibacterium | 7.50 |
| Sutterella | 3.28 | Faecalibacterium | 5.72 |
| Lactobacillus | 2.93 | Blautia | 5.43 |
| Paraprevotella | 1.83 | Clostridium | 4.20 |
| Species | Frequency (%) | Species | Frequency (%) |
| Unclassified at Species | 40.81 | Unclassified at Species | 36.16 |
| level | | level | |
| Fusobacterium | 22.88 | Prevotella copri | 18.27 |
| gonidiaformans | | | |
| Prevotella copri | 7.64 | Fusobacterium | 11.00 |
| | | gonidiaformans | |
| Catenibacterium | 3.18 | Clostridium hiranonis | 3.56 |
| mitsuokai | | | |
| Sutterella stercoricanis | 2.27 | Sutterella stercoricanis | 2.33 |
| Lactobacillus iners | 2.21 | Bacteroides coprocola | 2.26 |
| Paraprevotella clara | 1.82 | Bacteroides | 2.22 |
| | | xylanisolvens | |
| Halothiobacillus | 1.63 | Bacteroides | 1.76 |
| halophilus | | denticanum | |

TABLE 2.6
Top eight phyla, class, order, family, genus and species with estimates
of the relative frequencies (percentage of total hits) for dog
6 (Kiwi) before and after administration of EquiNectar ™.

| Before EquiNectar ™ | | After EquiNectar ™ | |
|---|---|---|---|
| Phylum | Frequency (%) | Phylum | Frequency (%) |
| Bacteroidetes | 37.08 | Bacteroidetes | 41.73 |
| Fusobacteria | 35.39 | Firmicutes | 30.56 |
| Firmicutes | 16.38 | Fusobacteria | 20.18 |
| Proteobacteria | 7.16 | Proteobacteria | 4.80 |
| Unclassified at Phylum | 3.20 | Unclassified at Phylum | 1.96 |
| level | | level | |
| Actinobacteria | 0.22 | Actinobacteria | 0.34 |
| Tenericutes | 0.16 | Tenericutes | 0.11 |
| Spirochaetes | 0.12 | Cyanobacteria | 0.09 |
| Class | Frequency (%) | Class | Frequency (%) |
| Bacteroidia | 36.41 | Bacteroidia | 40.78 |
| Fusobacteria | 35.39 | Clostridia | 23.36 |
| Clostridia | 12.06 | Fusobacteria | 20.18 |
| Betaproteobacteria | 4.76 | Erysipelotrichi | 6.17 |
| Unclassified at Class | 4.41 | Betaproteobacteria | 3.47 |
| level | | | |
| Erysipelotrichi | 2.84 | Unclassified at Class | 2.76 |
| | | level | |
| Bacilli | 1.38 | Bacilli | 0.87 |
| Gammaproteobacteria | 0.70 | Flavobacteriia | 0.62 |
| Order | Frequency (%) | Order | Frequency (%) |
| Bacteroidales | 36.41 | Bacteroidales | 40.78 |
| Fusobacteriales | 35.39 | Fusobacteriales | 20.18 |
| Clostridiales | 11.03 | Clostridiales | 19.90 |
| Burkholderiales | 4.72 | Erysipelotrichales | 6.17 |
| Unclassified at Order | 4.59 | Burkholderiales | 3.41 |
| level | | | |
| Erysipelotrichales | 2.84 | Coribacteriales | 3.39 |
| Lactobacillales | 1.09 | Unclassified at Order | 3.00 |
| | | level | |
| Coriobacteriales | 0.99 | Flavobacteriales | 0.62 |
| Family | Frequency (%) | Family | Frequency (%) |
| Fusobacteriaceae | 35.31 | Fusobacteriaceae | 20.10 |
| Prevotellaceae | 16.71 | Prevotellaceae | 19.04 |
| Bacteroidaceae | 11.72 | Bacteroidaceae | 13.17 |
| Paraprevotellaceae | 7.08 | Paraprevotellaceae | 7.26 |
| Unclassified at Family | 6.00 | Veillonellaceae | 5.80 |
| level | | | |
| Alcaligenaceae | 4.63 | Lachnospiraceae | 5.54 |
| Lachnospiraceae | 3.98 | Unclassified at Family | 4.98 |
| | | level | |
| Veillonellaceae | 3.57 | Ruminococcaceae | 4.78 |
| Genus | Frequency (%) | Genus | Frequency (%) |
| Fusobacterium | 29.00 | Prevotella | 20.47 |
| Prevotella | 18.37 | Fusobacterium | 15.54 |
| Bacteroides | 11.72 | Bacteroides | 13.17 |
| Unclassified at Genus | 9.79 | Unclassified at Genus | 8.21 |
| level | | level | |
| Paraprevotella | 5.42 | Paraprevotella | 5.83 |
| Sutterella | 4.63 | Faecalibacterium | 4.18 |
| Cetobacterium | 2.54 | Blautia | 3.88 |
| Blautia | 1.82 | Catenibacterium | 3.49 |

| Species | Frequency (%) | Species | Frequency (%) |
|---|---|---|---|
| Fusobacterium | 28.58 | Unclassified at | 29.90 |
| gonidiaformans | | Species level | |
| Unclassified at | 22.28 | Prevotella copri | 18.54 |
| Species level | | | |
| Prevotella copri | 16.44 | Fusobacterium | 15.25 |
| | | gonidiaformans | |
| Paraprevotella clara | 5.42 | Paraprevotella clara | 5.83 |
| Sutterella | 2.94 | Collinsella intestinalis | 2.82 |
| stercoricanis | | | |
| Bacteroides | 2.76 | Bacteroides | 2.21 |
| denticanum | | xylanisolvens | |
| Cetobacterium ceti | 2.54 | Clostridium hiranonis | 2.09 |
| Bacteroides | 2.42 | Bacteroides | 2.00 |
| coprocola | | denticanum | |

TABLE 2.7
Top eight phyla, class, order, family, genus and species with estimates
of the relative frequencies (percentage of total hits) for dog
7 (Jake) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 41.09 | Bacteroidetes | 47.04 |
| Bacteroidetes | 24.79 | Firmicutes | 36.65 |
| Fusobacteria | 22.09 | Fusobacteria | 7.82 |
| Proteobacteria | 9.06 | Proteobacteria | 5.58 |
| Unclassified at Phylum | 2.05 | Unclassified at | 1.95 |
| level | | Phylum level | |
| Actinobacteria | 0.20 | Actinobacteria | 0.43 |
| Verrucomicrobia | 0.17 | Cyanobacteria | 0.17 |
| Spirochaetes | 0.16 | Tenericutes | 0.11 |

| Class | Frequency (%) | Class | Frequency (%) |
|---|---|---|---|
| Clostridia | 32.04 | Bacteroidia | 42.81 |
| Bacteroidia | 23.14 | Clostridia | 25.92 |
| Fusobacteria | 22.09 | Erysipelotrichi | 9.42 |
| Betaproteobacteria | 6.11 | Fusobacteria | 7.82 |
| Bacilli | 5.10 | Flavobacteria | 3.46 |
| Erysipelotrichi | 3.81 | Unclassified at class | 3.29 |
| | | level | |
| Unclassified at class | 2.79 | Betaproteobacteria | 2.21 |
| level | | | |
| Gammaproteobacteria | 1.69 | Epsilonproteobacteria | 1.31 |
| Order | Frequency (%) | Order | Frequency (%) |
| Clostridiales | 30.68 | Bacteroidales | 42.81 |
| Bacteroidales | 23.14 | Clostridiales | 24.15 |
| Fusobacteriales | 22.09 | Erysipelotrichales | 9.42 |
| Burkholderiales | 6.04 | Fusobacteriales | 7.82 |
| Lactobacillus | 4.77 | Unclassified at Order | 3.56 |
| | | level | |
| Erysipelotrichales | 3.81 | Flavobacteriales | 3.46 |
| Unclassified at Order | 3.04 | Burkholderiales | 2.16 |
| level | | | |
| Aeromonadales | 1.37 | Coriobacteriales | 1.67 |

| Family | Frequency (%) | Family | Frequency (%) |
|---|---|---|---|
| Fusobacteriaceae | 22.05 | Bacteroidaceae | 17.07 |
| Veillonellaceae | 11.78 | Prevotellaceae | 14.59 |
| Bacteroidaceae | 9.25 | Paraprevotellaceae | 9.09 |
| Clostridiaceae | 8.66 | Coprobacillaceae | 7.98 |
| Prevotellaceae | 7.54 | Fusobacteriaceae | 7.78 |
| Lachnospiraceae | 7.54 | Lachnospiraceae | 7.42 |
| Alcaligenaceae | 5.95 | Veillonellaceae | 7.10 |
| Paraprevotellaceae | 5.86 | Unclassified at family | 5.93 |
| | | level | |

| Genus | Frequency (%) | Genus | Frequency (%) |
|---|---|---|---|
| Fusobacterium | 18.75 | Prevotella | 23.54 |
| Prevotella | 13.37 | Bacteroides | 17.07 |
| Megamonas | 10.31 | Catenibacterium | 7.83 |
| Bacteroides | 9.25 | Unclassified at Genus | 7.23 |
| | | level | |
| Clostridium | 7.61 | Fusobacterium | 6.65 |
| Sutterella | 5.95 | Blautia | 5.54 |
| Blautia | 5.81 | Flavobacterium | 3.27 |
| Unclassified at Genus | 5.78 | Faecalibacterium | 3.18 |
| level | | | |
| Species | Frequency (%) | Species | Frequency (%) |
| Unclassified at Species | 35.97 | Unclassified at | 44.92 |
| level | | Species level | |
| Fusobacterium | 18.31 | Prevotella copri | 13.41 |
| gonidaformans | | | |
| Prevotella copri | 7.15 | Fusobacterium | 6.38 |
| | | gonidaformans | |
| Clostridium hiranonis | 5.32 | Bacteroides | 3.96 |
| | | denticanum | |
| Bacteroides plebeius | 3.72 | Bacteroides | 2.68 |
| | | coprocola | |
| Megamonas | 3.03 | Phascolarcbacterium | 2.40 |
| funiformis | | succinatutens | |
| Streptococcus bovis | 2.39 | Bacteroides plebeius | 1.99 |
| Cetobacterium ceti | 2.31 | Clostridium hiranonis | 1.92 |

TABLE 2.8
Top eight phyla, class, order, family, genus and species with estimates
of the relative frequencies (percentage of total hits) for dog
8 (Hetty) before and after administration of EquiNectar ™.

| Before EquiNectar ™ | | After EquiNectar ™ | |
|---|---|---|---|
| Phylum | Frequency (%) | Phylum | Frequency (%) |
| Bacteroidetes | 50.14 | Bacteroidetes | 41.02 |
| Fusobacteria | 21.96 | Firmicutes | 26.89 |
| Firmicutes | 20.74 | Fusobacteria | 23.97 |
| Proteobacteria | 4.76 | Proteobacteria | 4.58 |
| Unclassified at Phylum | 1.68 | Unclassified at | 2.36 |
| level | | Phylum level | |
| Tenericutes | 0.27 | Actinobacteria | 0.31 |
| Actinobacteria | 0.20 | Tenericutes | 0.28 |
| Cyanobacteria | 0.08 | Cyanobacteria | 0.14 |

| Class | Frequency (%) | Class | Frequency (%) |
|---|---|---|---|
| Bacteroidia | 49.20 | Bacteroidia | 38.41 |
| Fusobacteria | 21.96 | Fusobacteria | 23.97 |
| Clostridia | 19.05 | Clostridia | 21.32 |
| Betaproteobacteria | 2.80 | Erysipelotrichi | 4.47 |
| Unclassified at Class | 2.28 | Unclassified at Class | 3.63 |
| level | | level | |
| Erysipelotrichi | 1.09 | Betaproteobacteria | 2.51 |
| Gammaproteobacteria | 0.78 | Flavobacteriia | 1.79 |
| Epsilonproteobacteria | 0.69 | Bacilli | 0.89 |
| Order | Frequency (%) | Order | Frequency (%) |
| Bacteroidales | 49.20 | Bacteroidales | 38.41 |
| Fusobacteriales | 21.96 | Fusobacteriales | 23.97 |
| Clostridiales | 18.37 | Clostridiales | 20.22 |
| Burkholderiales | 2.76 | Erysipelotrichales | 4.47 |
| Unclassified at Order | 2.44 | Unclassified at Order | 3.88 |
| level | | level | |
| Erysipelotrichales | 1.09 | Burkholderiales | 2.46 |
| Campylobacterales | 0.69 | Flavobacteriales | 1.79 |
| Coriobacteriales | 0.64 | Coriobacteriales | 1.03 |

| Family | Frequency (%) | Family | Frequency (%) |
|---|---|---|---|
| Bacteroidaceae | 33.05 | Fusobacteriaceae | 23.85 |
| Fusobacteriaceae | 21.89 | Bacteroidaceae | 20.49 |
| Prevotellaceae | 8.20 | Prevotellaceae | 10.07 |
| Lachnospiraceae | 7.84 | Lachnospiraceae | 7.62 |
| Paraprevotellaceae | 6.92 | Paraprevotellaceae | 6.58 |
| Unclassified at family | 3.99 | Unclassified at | 6.11 |
| level | | family level | |
| Veillonellaceae | 3.98 | Veillonellaceae | 5.16 |
| Clostridiaceae | 3.74 | Ruminococcaceae | 3.50 |

| Genus | Frequency (%) | Genus | Frequency (%) |
|---|---|---|---|
| Bacteroides | 33.05 | Bacteroides | 20.49 |
| Fusobacterium | 17.19 | Fusobacterium | 17.96 |
| Prevotella | 15.08 | Prevotella | 16.48 |
| Unclassified at Genus | 7.40 | Unclassified at | 10.27 |
| level | | Genus level | |
| Blautia | 5.21 | Blautia | 5.74 |
| Sutterella | 2.68 | Faecalibacterium | 2.97 |
| Clostridium | 2.55 | Megamonas | 2.81 |
| Phascolarctobacterium | 2.33 | Sutterella | 2.38 |

| Species | Frequency (%) | Species | Frequency (%) |
|---|---|---|---|
| Unclassified at species | 32.20 | Unclassified at | 36.78 |
| level | | species level | |
| Fusobacterium | 16.93 | Fusobacterium | 17.56 |
| gonidiaformans | | gonidiaformans | |
| Bacteroides | 12.80 | Prevotella copri | 9.18 |
| denticanum | | | |
| Prevotella copri | 7.54 | Bacteroides | 7.07 |
| | | denticanum | |
| Bacteroides rodentium | 6.87 | Bacteroides | 3.02 |
| | | rodentium | |
| Bacteroides coprocola | 3.89 | Blautia coccoides | 1.89 |
| Clostridium hiranonis | 2.15 | Cetobacterium ceti | 1.76 |
| Blautia coccoides | 1.70 | Bacteroides | 1.74 |
| | | coprocola | |

TABLE 2.9
Top eight phyla, class, order, family, genus and species with estimates
of the relative frequencies (percentage of total hits) for dog 9
(Gunner) before and after administration of EquiNectar ™.

| Before EquiNectar ™ | | After EquiNectar ™ | |
|---|---|---|---|
| Phylum | Frequency (%) | Phylum | Frequency (%) |
| Bacteroidetes | 35.84 | Bacteroidetes | 41.42 |
| Firmicutes | 29.97 | Firmicutes | 30.05 |
| Fusobacteria | 25.13 | Fusobacteria | 21.52 |
| Proteobacteria | 5.88 | Proteobacteria | 4.10 |
| Unclassified at Phylum | 2.39 | Unclassified at Phylum | 1.99 |
| level | | level | |
| Actinobacteria | 0.28 | Tenericutes | 0.29 |
| Tenericutes | 0.19 | Actinobacteria | 0.23 |
| Cyanobacteria | 0.14 | Cyanobacteria | 0.11 |
| Class | Frequency (%) | Class | Frequency (%) |
| Bacteroidia | 35.19 | Bacteroidia | 39.14 |
| Clostridia | 25.61 | Clostridia | 25.70 |
| Fusobacteria | 25.13 | Fusobacteria | 21.52 |
| Unclassified at Class | 3.25 | Erysipelotrichi | 3.32 |
| level | | | |
| Erysipelotrichi | 3.10 | Unclassified at Class | 2.84 |
| | | level | |
| Betaproteobacteria | 2.78 | Betaproteobacteria | 2.38 |
| Gammaproteobacteria | 1.37 | Flavobacteriia | 1.68 |
| Bacilli | 1.05 | Bacilli | 0.90 |
| Order | Frequency (%) | Order | Frequency (%) |
| Bacteroidales | 35.19 | Bacteroidales | 39.14 |
| Fusobacteriales | 25.13 | Clostridiales | 24.35 |
| Clostridiales | 24.18 | Fusobacteriales | 21.52 |
| Unclassified at Order | 3.50 | Erysipelotrichales | 3.32 |
| level | | | |
| Erysipelotrichales | 3.10 | Unclassified at Order | 3.05 |
| | | level | |
| Burkholderiales | 2.74 | Burkholderiales | 2.36 |
| Coriobacteriales | 1.36 | Flavobacteriales | 1.68 |
| Aeromonadales | 1.05 | Coriobacteriales | 1.26 |
| Family | Frequency (%) | Family | Frequency (%) |
| Fusobacteriaceae | 25.04 | Fusobacteriaceae | 21.46 |
| Bacteroidaceae | 18.01 | Bacteroidaceae | 17.97 |
| Prevotellaceae | 10.75 | Prevotellaceae | 13.88 |
| Lachnospiraceae | 9.64 | Lachnospiraceae | 10.72 |
| Veillonellaceae | 7.09 | Paraprevotellaceae | 6.19 |
| Paraprevotellaceae | 5.46 | Veillonellaceae | 5.20 |
| Unclassified at Family | 5.36 | Unclassified at Family | 5.05 |
| level | | level | |
| Clostridiaceae | 4.68 | Clostridiaceae | 4.79 |
| Genus | Frequency (%) | Genus | Frequency (%) |
| Fusobacterium | 19.61 | Prevotella | 20.02 |
| Bacteroides | 18.01 | Bacteroides | 17.97 |
| Prevotella | 16.16 | Fusobacterium | 16.35 |
| Unclassified at Genus | 9.41 | Unclassified at Genus | 8.62 |
| level | | level | |
| Blautia | 6.37 | Blautia | 6.62 |
| Phascolarctobacterium | 4.70 | Clostridium | 3.56 |
| Clostridium | 3.86 | Phascolarctobacterium | 3.18 |
| Sutterella | 2.68 | Lachnospira | 2.30 |
| Species | Frequency (%) | Species | Frequency (%) |
| Unclassified at Species | 31.94 | Unclassified at Species | 32.61 |
| level | | level | |
| Fusobacterium | 19.31 | Fusobacterium | 15.98 |
| gonidiaformans | | gonidiaformans | |
| Prevotella copri | 10.00 | Prevotella copri | 12.90 |
| Bacteroides | 5.07 | Bacteroides rodentium | 4.04 |
| denticanum | | | |
| Bacteroides coprocola | 4.33 | Bacteroides | 3.20 |
| | | denticanum | |
| Clostridium hiranonis | 3.26 | Clostridium hiranonis | 2.85 |
| Bacteroides rodentium | 3.13 | Bacteroides coprocola | 2.51 |
| Blautia coccoides | 2.22 | Blautia coccoides | 2.43 |

TABLE 2.10
Top eight phyla, class, order, family, genus and species with estimates
of the relative frequencies (percentage of total hits) for dog 10
(Digby) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 49.68 | Bacteroidetes | 56.73 |
| Bacteroidetes | 29.18 | Firmicutes | 29.18 |
| Proteobacteria | 11.12 | Fibrobacteres | 3.61 |
| Fusobacteria | 7.63 | Spirochaetes | 3.55 |
| Unclassified at Phylum | 1.67 | Proteobacteria | 3.06 |
| level | | | |
| Actinobacteria | 0.25 | Unclassified at | 0.94 |
| | | Phylum level | |
| Tenericutes | 0.14 | Fusobacteria | 0.91 |
| Cyanobacteria | 0.12 | Tenericutes | 0.76 |

| Class | Frequency (%) | Class | Frequency (%) |
|---|---|---|---|
| Clostridia | 36.20 | Bacteroidia | 55.65 |
| Bacteroidia | 28.76 | Clostridia | 24.58 |
| Erysipelotrichi | 11.87 | Fibrobacteria | 3.61 |
| Fusobacteria | 7.63 | Spirochaetia | 3.55 |
| Gammaproteobacteria | 6.72 | Erysipelotrichia | 3.12 |
| Unclassified at Class | 2.55 | Unclassified at Class | 1.69 |
| level | | level | |
| Betaproteobacteria | 2.38 | Betaproteobacteria | 1.47 |
| Bacilli | 1.38 | Fusobacteriia | 0.91 |

| Order | Frequency (%) | Order | Frequency (%) |
|---|---|---|---|
| Clostridiales | 32.90 | Bacteroidales | 55.65 |
| Bacteroidales | 28.76 | Clostridiales | 24.34 |
| Erysipelotrichales | 11.87 | Fibrobacterales | 3.61 |
| Fusobacteriales | 7.63 | Spirochaetales | 3.55 |
| Aeromonadales | 6.08 | Erysipelotrichales | 3.12 |
| Coriobacteriales | 3.25 | Unclassified at | 1.84 |
| | | Order level | |
| Unclassified at Order | 2.92 | Burkholderiales | 1.46 |
| level | | | |
| Burkholderiales | 2.34 | Fusobacteriales | 0.91 |

| Family | Frequency (%) | Family | Frequency (%) |
|---|---|---|---|
| Prevotellaceae | 16.38 | Prevotellaceae | 46.22 |
| Lachnospiraceae | 11.52 | Ruminococcaceae | 16.90 |
| Veillonellaceae | 10.59 | Bacteroidaceae | 4.99 |
| Bacteroidaceae | 10.11 | Lachnospiraceae | 4.52 |
| Coprobacillaceae | 9.74 | Fibrobacteraceae | 3.61 |
| Fusobacteriaceae | 7.60 | Spirochaetaceae | 3.55 |
| Succinivibrionaceae | 6.00 | Unclassified at | 3.14 |
| | | Family level | |
| Clostridiaceae | 5.70 | Erysipelotrichaceae | 3.12 |

| Genus | Frequency (%) | Genus | Frequency (%) |
|---|---|---|---|
| Prevotella | 17.79 | Prevotella | 41.53 |
| Bacteroides | 10.11 | Faecalibacterium | 13.99 |
| Catenibacterium | 9.21 | Unclassified at | 5.87 |
| | | Genus level | |
| Unclassified at Genus | 7.51 | Bacteroides | 4.99 |
| level | | | |
| Blautia | 6.30 | Alloprevotella | 4.28 |
| Megamonas | 5.79 | Fibrobacter | 3.61 |
| Succinivibrio | 5.68 | Treponema | 3.52 |
| Fusobacterium | 4.62 | Parabacteroides | 1.66 |

| Species | Frequency (%) | Species | Frequency (%) |
|---|---|---|---|
| Unclassified at Species | 42.19 | Prevotella copri | 40.36 |
| level | | | |
| Prevotella copri | 16.07 | Unclassified at | 15.70 |
| | | Species level | |
| Fusobacterium | 4.55 | Faecalibacterium | 13.99 |
| gonidiaformans | | prausnitzii | |
| Clostridium hiranonis | 3.63 | Fibrobacter | 3.47 |
| | | succinogenes | |
| Bacteroides coprocola | 3.04 | Treponema bryantii | 2.59 |
| Collinsella intestinalis | 2.84 | Alloprevotella rava | 2.27 |
| Phascolarctobacterium | 2.35 | Prevotellamassilia | 1.79 |
| succinatutens | | timonensis | |
| Succinivibrio | 2.21 | Bacteroides plebeius | 1.13 |
| dextrinosolvens | | | |

TABLE 2.11
Top eight phyla, class, order, family, genus and species with estimates of the relative frequencies
(percentage of total hits) for dog 11 (Cody) before and after administration of EquiNectar ™.

| Before EquiNectar ™ | | After EquiNectar ™ | |
|---|---|---|---|
| Phylum | Frequency (%) | Phylum | Frequency (%) |
| Fusobacteria | 38.84 | Bacteroidetes | 47.05 |
| Firmicutes | 31.42 | Firmicutes | 23.68 |
| Bacteroidetes | 16.60 | Proteobacteria | 19.88 |
| Proteobacteria | 7.70 | Fusobacteria | 6.74 |
| Unclassified at Phylum level | 4.12 | Spirochaetes | 0.69 |
| Actinobacteria | 0.33 | Unclassified at Phylum level | 0.68 |
| Verrucomicrobia | 0.21 | Fibrobacteres | 0.47 |
| Spirochaetes | 0.20 | Tenericutes | 0.22 |
| Class | Frequency (%) | Class | Frequency (%) |
| Fusobacteria | 38.84 | Bacteroidia | 46.62 |
| Clostridia | 20.49 | Betaproteobacteria | 17.80 |
| Bacteroidia | 15.77 | Clostridia | 15.52 |
| Bacilli | 7.84 | Fusobacteriia | 6.74 |
| Unclassified at Class level | 5.45 | Negativicutes | 5.99 |
| Betaproteobacteria | 5.17 | Erysipelotrichia | 1.60 |
| Erysipelotrichi | 2.81 | Unclassified at Class level | 1.08 |
| Gammaproteobacteria | 1.25 | Gammaproteobacteria | 1.01 |
| Order | Frequency (%) | Order | Frequency (%) |
| Fusobacterials | 38.84 | Bacteroidales | 46.62 |
| Clostridiales | 19.10 | Burkholderiales | 17.72 |
| Bacteroidales | 15.77 | Clostridiales | 15.48 |
| Turicibacterales | 6.52 | Fusobacteriales | 6.74 |
| Unclassified at Order level | 5.80 | Selenomonadales | 5.99 |
| Burkholderiales | 5.09 | Erysipelotrichales | 1.60 |
| Erysipelotrichales | 2.81 | Unclassified at Order level | 1.17 |
| Coriobacteriales | 1.28 | Aeromonadales | 0.93 |
| Family | Frequency (%) | Family | Frequency (%) |
| Fusobacteriaceae | 38.77 | Prevotellaceae | 35.51 |
| Lachnospiraceae | 6.98 | Sutterellaceae | 17.62 |
| Unclassified at Family level | 6.90 | Ruminococcaceae | 12.69 |
| Turicibacteraceae | 6.52 | Bacteroidaceae | 9.58 |
| Clostridiaceae | 6.38 | Fusobacteriaceae | 6.74 |
| Paraprevotellaceae | 5.45 | Acidaminococcaceae | 5.78 |
| Prevotellaceae | 5.39 | Lachnospiraceae | 1.94 |
| Alcaligenaceae | 5.03 | Unclassified at Family level | 1.88 |
| Genus | Frequency (%) | Genus | Frequency (%) |
| Fusobacterium | 32.66 | Prevotella | 33.62 |
| Unclassified at Genus level | 10.89 | Sutterella | 16.95 |
| Prevotella | 6.95 | Faecalibacterium | 11.49 |
| Turicibacter | 6.52 | Bacteroides | 9.58 |
| Clostridium | 5.04 | Fusobacterium | 6.55 |
| Sutterella | 5.02 | Phascolarctobacterium | 5.74 |
| Bacteroides | 4.30 | Unclassified at Genus level | 3.22 |
| Lachnospira | 3.94 | Alloprevotella | 1.18 |
| Species | Frequency (%) | Species | Frequency (%) |
| Unclassified at Species level | 33.97 | Prevotella copri | 32.93 |
| Fusobacterium gonidiaformans | 31.79 | Unclassified at Species level | 12.61 |
| Prevotella copri | 5.13 | Faecalibacterium prausnitzii | 11.49 |
| Paraprevotella clara | 3.89 | Sutterella stercoricanis | 9.72 |
| Sutterella stercoricanis | 3.11 | Phascolarctobacterium succinatutens | 5.67 |
| Lachnospira pectinoschiza | 2.95 | Sutterella massiliensis | 5.17 |
| Cetobacterium ceti | 2.65 | Bacteroides plebeius | 5.04 |
| Turicibacter sanguinis | 1.18 | Fusobacterium perfoetens | 3.93 |

TABLE 2.12
Top eight phyla, class, order, family, genus and species with estimates of the relative frequencies
(percentage of total hits) for dog 12 (Chip) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 64.90 | Firmicutes | 59.38 |
| Bacteroidetes | 10.01 | Bacteroidetes | 21.56 |
| Proteobacteria | 9.69 | Fusobacteria | 7.85 |
| Fusobacteria | 9.08 | Proteobacteria | 5.20 |
| Unclassified at Phylum level | 2.29 | Actinobacteria | 3.65 |
| Verrucomicrobia | 1.13 | Unclassified at Phylum level | 1.75 |
| Spirochaetes | 1.04 | Tenericutes | 0.20 |
| Fibrobacteres | 0.45 | Cyanobacteria | 0.13 |
| Class | Frequency (%) | Class | Frequency (%) |
| Clostridia | 50.87 | Clostridia | 35.25 |
| Fusobacteria | 9.08 | Bacteroidia | 20.05 |
| Gammaproteobacteria | 8.20 | Erysipelotrichi | 13.71 |
| Erysipelotrichi | 7.65 | Bacilli | 10.09 |
| Bacteroidia | 7.31 | Fusobacteria | 7.85 |
| Bacilli | 5.88 | Actinobacteria | 3.64 |
| Unclassified at Class level | 3.84 | Gammaproteobacteria | 3.13 |
| Sphingobacteriia | 1.87 | Unclassified at Class level | 2.73 |
| Order | Frequency (%) | Order | Frequency (%) |
| Clostridiales | 43.57 | Clostridiales | 31.04 |
| Fusobacteriales | 9.08 | Bacteroidales | 20.05 |
| Erysipelotrichales | 7.65 | Erysipelotrichales | 13.71 |
| Bacteroidales | 7.31 | Lactobacillales | 9.64 |
| Coriobacteriales | 7.06 | Fusobacteriales | 7.85 |
| Aeromonadales | 5.57 | Coriobacteriales | 4.11 |
| Unclassified at Order level | 4.47 | Bifidobacteriales | 3.26 |
| Turicibacterales | 2.95 | Unclassified at Order level | 3.13 |
| Family | Frequency (%) | Family | Frequency (%) |
| Lachnospiraceae | 20.03 | Lachnospiraceae | 14.68 |
| Fusobacteriaceae | 9.06 | Coprobacillaceae | 11.51 |
| Clostridiaceae | 7.30 | Lactobacillaceae | 8.74 |
| Coriobacteriaceae | 7.06 | Bacteroidaceae | 8.42 |
| Unclassified at Family level | 6.42 | Fusobacteriaceae | 7.83 |
| Veillonellaceae | 5.56 | Veillonellaceae | 7.12 |
| Succinivibrionaceae | 5.51 | Clostridiaceae | 6.00 |
| Ruminococcaceae | 4.49 | Paraprevotellaceae | 5.75 |
| Genus | Frequency (%) | Genus | Frequency (%) |
| Blautia | 13.87 | Blautia | 11.32 |
| Unclassified at Genus level | 9.10 | Catenibacterium | 11.09 |
| Fusobacterium | 7.91 | Prevotella | 9.25 |
| Clostridium | 7.38 | Lactobacillus | 8.52 |
| Collinsella | 6.12 | Bacteroides | 8.42 |
| Succinivibrio | 5.47 | Fusobacterium | 6.53 |
| Ruminococcus | 5.46 | Unclassified at Genus level | 6.53 |
| Catenibacterium | 3.67 | Clostridium | 5.00 |
| Species | Frequency (%) | Species | Frequency (%) |
| Unclassified at Species level | 49.44 | Unclassified at Species level | 38.87 |
| Fusobacterium gonidiaformans | 7.67 | Fusobacterium gonidiaformans | 6.34 |
| Collinsella intestinalis | 5.58 | Lactobacillus hayakitensis | 5.21 |
| Blautia coccoides | 3.36 | Prevotella copri | 4.93 |
| Prevotella copri | 2.83 | Clostridium hiranonis | 3.16 |
| Eubacterium biforme | 2.31 | Blautia coccoides | 2.72 |
| Succinivibrio dextrinosolvens | 2.27 | Bacteroides coprocola | 2.23 |
| Phascolarctobacterium succinatutens | 1.62 | Collinsella intestinalis | 2.00 |

TABLE 2.13
Top eight phyla, class, order, family, genus and species with estimates
of the relative frequencies (percentage of total hits) for dog 13 (Bumper)
before and after administration of EquiNectar^{T}).

| Before EquiNectar ™ | | After EquiNectar ™ | |
|---|---|---|---|
| Phylum | Frequency (%) | Phylum | Frequency (%) |
| Firmicutes | 72.27 | Firmicutes | 47.09 |
| Bacteroidetes | 12.22 | Bacteroidetes | 37.08 |
| Proteobacteria | 12.10 | Proteobacteria | 9.76 |
| Unclassified at Phylum level | 1.55 | Fusobacteria | 3.51 |
| Fusobacteria | 0.69 | Unclassified at Phylum level | 1.55 |
| Actinobacteria | 0.55 | Actinobacteria | 0.61 |
| Cyanobacteria | 0.21 | Cyanobacteria | 0.15 |
| Tenericutes | 0.09 | Tenericutes | 0.06 |
| Class | Frequency (%) | Class | Frequency (%) |
| Clostridia | 51.89 | Clostridia | 32.76 |
| Erysipelotrichi | 18.15 | Bacteroidia | 32.56 |
| Bacteroidia | 11.55 | Erysipelotrichi | 13.16 |
| Gammaproteobacteria | 10.72 | Gammaproteobacteria | 4.73 |
| Unclassified at Class level | 2.75 | Flavobacteriia | 3.69 |
| Bacilli | 2.00 | Fusobacteria | 3.51 |
| Fusobacteria | 0.69 | Epsilonproteobacteria | 3.48 |
| Actinobacteria | 0.55 | Unclassified at Class level | 2.99 |
| Order | Frequency (%) | Order | Frequency (%) |
| Clostridiales | 48.01 | Bacteroidales | 32.56 |
| Erysipelotrichales | 18.15 | Clostridiales | 29.02 |
| Bacteroidales | 11.55 | Erysipelotrichales | 13.16 |
| Aeromonadales | 9.74 | Flavobacteriales | 3.69 |
| Coriobacteriales | 3.79 | Coriobacteriales | 3.66 |
| Unclassified at Order level | 3.16 | Aeromonadales | 3.62 |
| Turicibacterales | 0.91 | Fusobacteriales | 3.51 |
| Lactobacillies | 0.87 | Campylobacterales | 3.48 |
| Family | Frequency (%) | Family | Frequency (%) |
| Veillonellaceae | 36.25 | Veillonellaceae | 14.37 |
| Coprobacillaceae | 16.96 | Paraprevotellaceae | 12.86 |
| Succinivibrionaceae | 9.66 | Prevotellaceae | 12.84 |
| Lachnospiraceae | 8.05 | Coprobacillaceae | 11.77 |
| Prevotellaceae | 7.86 | Lachnospiraceae | 9.70 |
| Unclassified at Family level | 4.56 | Bacteroidaceae | 6.23 |
| Coriobacteriaceae | 3.79 | Unclassified at Family level | 5.07 |
| Paraprevotellaceae | 2.11 | Flavobacteriaceae | 3.69 |
| Genus | Frequency (%) | Genus | Frequency (%) |
| Megasphaera | 22.30 | Prevotella | 25.59 |
| Catenibacterium | 16.94 | Megamonas | 12.49 |
| Megamonas | 13.18 | Catenibacterium | 11.75 |
| Prevotella | 9.92 | Unclassified at Genus level | 6.54 |
| Succinivibrio | 9.66 | Blautia | 6.48 |
| Unclassified at Genus level | 5.60 | Bacteroides | 6.23 |
| Ruminococcus | 4.08 | Flavobacterium | 3.54 |
| Blautia | 3.50 | Campylobacter | 3.45 |
| Species | Frequency (%) | Species | Frequency (%) |
| Unclassified at Species level | 46.38 | Unclassified at Species level | 52.33 |
| Megasphaera elsdenii | 13.45 | Prevotella copri | 11.43 |
| Megasphaera hominis | 8.52 | Megamonas funiformis | 4.40 |
| Prevotella copri | 7.63 | Fusobacterium gonidiaformans | 2.66 |
| Megamonas funiformis | 3.95 | Catenibacterium mitsuokai | 2.22 |
| Succinivibrio dextrinosolvens | 3.75 | Bacteroides coprocola | 2.12 |
| Catenibacterium mitsuokai | 2.78 | Campylobacter upsaliensis | 2.04 |
| Collinsella aerofaciens | 1.90 | Collinsella intestinalis | 1.90 |

TABLE 2.14
Top eight phyla, class, order, family, genus and species with estimates of the relative frequencies
(percentage of total hits) for dog 14 (Buddy) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 36.29 | Firmicutes | 66.79 |
| Bacteroidetes | 25.63 | Proteobacteria | 13.87 |
| Fusobacteria | 11.66 | Bacteroidetes | 10.36 |
| Proteobacteria | 6.71 | Fusobacteria | 5.92 |
| Verrucomicrobia | 4.46 | Unclassified at Phylum level | 1.99 |
| Spirochaetes | 4.17 | Actinobacteria | 0.30 |
| Fibrobacteres | 4.00 | Tenericutes | 0.26 |
| Unclassified at Phylum level | 3.92 | Cyanobacteria | 0.24 |
| Class | Frequency (%) | Class | Frequency (%) |
| Clostridia | 24.28 | Clostridia | 40.07 |
| Bacteroidia | 16.44 | Erysipelotrichi | 16.44 |
| Fusobacteria | 11.66 | Gammaproteobacteria | 12.26 |
| Erysipelotrichi | 9.58 | Bacilli | 9.97 |
| Unclassified at Class level | 7.51 | Bacteroidia | 9.83 |
| Sphingobacteriia | 6.60 | Fusobacteria | 5.92 |
| Gammaproteobacteria | 4.20 | Unclassified at Class level | 2.82 |
| Spirochaetes | 4.17 | Betaproteobacteria | 0.96 |
| Order | Frequency (%) | Order | Frequency (%) |
| Clostridiales | 22.49 | Clostridiales | 28.10 |
| Bacteroidales | 16.44 | Erysipelotrichales | 16.44 |
| Fusobacteriales | 11.66 | Coriobacteriales | 11.86 |
| Erysipelotrichales | 9.58 | Aeromonadales | 10.96 |
| Unclassified at Order level | 8.05 | Bacteroidales | 9.83 |
| Sphingobacteriales | 6.60 | Turicibacterales | 8.75 |
| Methylacidiphilales | 4.04 | Fusobacteriales | 5.92 |
| Fibrobacterales | 4.00 | Unclassified at Order level | 3.68 |
| Family | Frequency (%) | Family | Frequency (%) |
| Unclassified at Family level | 12.33 | Lachnospiraceae | 12.24 |
| Fusobacteriaceae | 11.63 | Coriobacteriaceae | 11.86 |
| Coprobacillaceae | 7.95 | Succinivibrionaceae | 10.80 |
| Lachnospiraceae | 7.24 | Coprobacillaceae | 9.15 |
| Clostridiaceae | 6.67 | Turicibacteraceae | 8.75 |
| Sphingobacteriaceae | 5.91 | Erysipelotrichaceae | 7.25 |
| Prevotellaceae | 4.87 | Prevotellaceae | 6.99 |
| Ruminococcaceae | 4.72 | Fusobacteriaceae | 5.90 |
| Genus | Frequency (%) | Genus | Frequency (%) |
| Unclassified at Genus level | 16.74 | Succinivibrio | 10.80 |
| Fusobacterium | 10.32 | Collinsella | 10.68 |
| Catenibacterium | 7.93 | Turicibacter | 8.75 |
| Prevotella | 5.30 | Catenibacterium | 8.70 |
| Blautia | 4.22 | Prevotella | 7.72 |
| Candidatus Methylacidiphilum | 4.04 | Unclassified at Genus level | 6.59 |
| Fibrobacter | 4.00 | Blautia | 6.58 |
| Treponema | 3.97 | Eubacterium | 6.24 |
| Species | Frequency (%) | Species | Frequency (%) |
| Unclassified at Species level | 48.24 | Unclassified at Species level | 42.94 |
| Fusobacterium gonidiaformans | 10.10 | Collinsella intestinalis | 8.24 |
| Sedimentibacter hydroxybenzoicus | 3.40 | Prevotella copri | 6.54 |
| Sphingobacterium bambusae | 3.31 | Succinivibrio dextrinosolvens | 5.87 |
| Fibrobacter succinogenes | 3.22 | Eubacterium biforme | 5.87 |
| Prevotella copri | 3.00 | Fusobacterium gonidiaformans | 5.05 |
| Dysgonomonas wimpennyi | 1.70 | Clostridium hiranonis | 3.40 |
| Treponema bryantii | 1.48 | Turicibacter sanguinis | 2.58 |

TABLE 2.15
Top eight phyla, class, order, family, genus and species with estimates of the relative frequencies
(percentage of total hits) for dog 15 (Bob) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 35.83 | Firmicutes | 41.86 |
| Bacteroidetes | 32.52 | Bacteroidetes | 28.97 |
| Fusobacteria | 7.23 | Unclassified at Phylum level | 4.99 |
| Proteobacteria | 5.67 | Proteobacteria | 4.48 |
| Spirochaetes | 4.80 | Spirochaetes | 4.40 |
| Unclassified at Phylum level | 4.00 | Fusobacteria | 4.20 |
| Fibrobacteres | 3.47 | Verrucomicrobia | 4.08 |
| Verrucomicrobia | 3.21 | Fibrobacteres | 2.76 |
| Class | Frequency (%) | Class | Frequency (%) |
| Clostridia | 28.60 | Clostridia | 34.17 |
| Bacteroidia | 22.72 | Bacteroidia | 18.77 |
| Fusobacteria | 7.23 | Unclassified at Class level | 8.69 |
| Unclassified at Class level | 7.16 | Sphingobacteriia | 6.87 |
| Sphingobacteriia | 7.16 | Erysipelotrichi | 4.92 |
| Spirochaetes | 4.79 | Spirochaetes | 4.40 |
| Erysipelotrichi | 4.48 | Fusobacteria | 4.20 |
| Fibrobacteria | 3.47 | Methylacidiphilae | 3.34 |
| Order | Frequency (%) | Order | Frequency (%) |
| Clostridiales | 26.40 | Clostridiales | 23.92 |
| Bacteroidales | 22.72 | Bacteroidales | 18.77 |
| Unclassified at Order level | 7.87 | Unclassified at Order level | 9.57 |
| Fusobacteriales | 7.23 | Coriobacteriales | 9.30 |
| Sphingobacteriales | 7.16 | Sphingobacteriales | 6.87 |
| Spirochaetales | 4.61 | Erysipelotrichales | 4.92 |
| Erysipelotrichales | 4.48 | Spirochaetales | 4.30 |
| Fibrobacterales | 3.47 | Fusobacteriales | 4.20 |
| Family | Frequency (%) | Family | Frequency (%) |
| Lachnospiraceae | 11.39 | Unclassified at Family level | 14.11 |
| Unclassified at Family level | 11.36 | Coriobacteriaceae | 9.30 |
| Bacteroidaceae | 10.18 | Lachnospiraceae | 8.74 |
| Fusobacteriaceae | 7.20 | Clostridiaceae | 6.91 |
| Sphingobacteriaceae | 6.41 | Sphingobacteriaceae | 6.11 |
| Clostridiaceae | 6.19 | Bacteroidaceae | 5.43 |
| Ruminococcaceae | 5.02 | Porphyromonadaceae | 4.98 |
| Porphyromonadaceae | 4.98 | Spirochaetaceae | 4.30 |
| Genus | Frequency (%) | Genus | Frequency (%) |
| Unclassified at Genus level | 16.51 | Unclassified at Genus level | 18.84 |
| Bacteroides | 10.18 | Collinsella | 7.70 |
| Blautia | 6.07 | Blautia | 6.09 |
| Fusobacterium | 5.86 | Prevotella | 5.58 |
| Prevotella | 4.72 | Bacteroides | 5.43 |
| Treponema | 4.61 | Clostridium | 4.81 |
| Clostridium | 3.85 | Treponema | 4.30 |
| Fibrobacter | 3.47 | Fusobacterium | 3.53 |
| Species | Frequency (%) | Species | Frequency (%) |
| Unclassified at Species level | 46.31 | Unclassified at Species level | 49.19 |
| Fusobacterium gonidiaformans | 5.73 | Collinsella intestinalis | 7.50 |
| Bacteroides denticanum | 4.29 | Fusobacterium gonidiaformans | 3.38 |
| Fibrobacter succinogenes | 3.39 | Prevotella copri | 2.78 |
| Sphingobacterium bambusae | 2.87 | Fibrobacter succinogenes | 2.71 |
| Dysgonomonas wimpennyi | 2.79 | Dysgonomonas wimpennyi | 2.34 |
| Lachnospira pectinoschiza | 2.54 | Treponema bryantii | 2.12 |
| Treponema bryantii | 2.51 | Sphingobacterium bambusae | 1.87 |

TABLE 2.16
Top eight phyla, class, order, family, genus and species with estimates of the relative frequencies
(percentage of total hits) for dog 16 (Belle) before and after administration of EquiNectar ™.

| Before EquiNectar ™ | | After EquiNectar ™ | |
|---|---|---|---|
| Phylum | Frequency (%) | Phylum | Frequency (%) |
| Bacteroidetes | 38.18 | Bacteroidetes | 48.82 |
| Fusobacteria | 34.24 | Firmicutes | 22.89 |
| Firmicutes | 18.06 | Fusobacteria | 17.91 |
| Proteobacteria | 5.10 | Proteobacteria | 7.80 |
| Unclassified at Phylum level | 3.39 | Unclassified at Phylum level | 1.61 |
| Actinobacteria | 0.39 | Actinobacteria | 0.41 |
| Deferribacteres | 0.16 | Spirochaetes | 0.12 |
| Spirochaetes | 0.11 | Cyanobacteria | 0.11 |
| Class | Frequency (%) | Class | Frequency (%) |
| Bacteroidia | 37.16 | Bacteroidia | 46.42 |
| Fusobacteria | 34.24 | Clostridia | 19.96 |
| Clostridia | 15.16 | Fusobacteria | 17.91 |
| Unclassified at Class level | 4.62 | Betaproteobacteria | 3.92 |
| Betaproteobacteria | 1.97 | Gammaproteobacteria | 2.89 |
| Bacilli | 1.74 | Unclassified at Class level | 2.53 |
| Erysipelotrichi | 1.05 | Erysipelotrichi | 1.93 |
| Epsilonproteobacteria | 1.04 | Flavobacteriia | 1.68 |
| Order | Frequency (%) | Order | Frequency (%) |
| Bacteroidales | 37.16 | Bacteroidales | 46.42 |
| Fusobacteriales | 34.24 | Clostridiales | 19.05 |
| Clostridiales | 14.91 | Fusobacteriales | 17.97 |
| Unclassified at Order level | 4.86 | Burkholderiales | 3.86 |
| Burkholderiales | 1.94 | Unclassified at Order level | 2.80 |
| Erysipelotrichales | 1.05 | Aeromonadales | 2.51 |
| Campylobacterales | 1.04 | Erysipelotrichales | 1.93 |
| Lactobacillies | 0.85 | Flavobacteriales | 1.68 |
| Family | Frequency (%) | Family | Frequency (%) |
| Fusobacteriaceae | 34.16 | Bacteroidaceae | 29.86 |
| Bacteroidaceae | 21.24 | Fusobacteriaceae | 17.85 |
| Prevotellaceae | 9.29 | Prevotellaceae | 9.30 |
| Veillonellaceae | 6.48 | Paraprevotellaceae | 6.11 |
| Unclassified at Family level | 6.37 | Lachnospiraceae | 5.73 |
| Paraprevotellaceae | 5.64 | Ruminococcaceae | 5.12 |
| Lachnospiraceae | 4.73 | Unclassified at Family level | 4.29 |
| Alcaligenaceae | 1.89 | Veillonellaceae | 3.87 |
| Genus | Frequency (%) | Genus | Frequency (%) |
| Fusobacterium | 26.69 | Bacteroides | 29.86 |
| Bacteroides | 21.24 | Prevotella | 15.36 |
| Prevotella | 14.87 | Fusobacterium | 13.14 |
| Unclassified at Genus level | 11.16 | Unclassified at Genus level | 8.10 |
| Megamonas | 4.27 | Faecalibacterium | 4.54 |
| Blauti | 3.53 | Blautia | 3.86 |
| Cetobacterium | 2.69 | Sutterella | 3.75 |
| Phascolarctobacterium | 1.93 | Anaerobiospirillum | 2.42 |
| Species | Frequency (%) | Species | Frequency (%) |
| Unclassified at Species level | 32.64 | Unclassified at Species level | 36.57 |
| Fusobacterium gonidiaformans | 26.30 | Fusobacterium gonidiaformans | 12.96 |
| Bacteroides denticanum | 8.85 | Bacteroides denticanum | 10.84 |
| Prevotella copri | 8.51 | Prevotella copri | 8.57 |
| Bacteroides coprocola | 3.47 | Bacteroides rodentium | 6.31 |
| Cetobacterium ceti | 2.69 | Anaerobiospirillum succiniciproducens | 2.37 |
| Bacteroides rodentium | 2.25 | Bacteroides coprocola | 1.93 |
| Megamonas funiformis | 1.31 | Clostridium hiranonis | 1.76 |

TABLE 2.17
Top eight phyla, class, order, family, genus and species with estimates of the relative frequencies
(percentage of total hits) for dog 17 (Bear) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 55.50 | Bacteroidetes | 40.73 |
| Bacteroidetes | 18.39 | Firmicutes | 24.75 |
| Fusobacteria | 17.05 | Fusobacteria | 24.72 |
| Proteobacteria | 4.99 | Proteobacteria | 7.02 |
| Unclassified at Phylum level | 2.31 | Unclassified at Phylum level | 1.96 |
| Actinobacteria | 0.51 | Actinobacteria | 0.30 |
| Spirochaetes | 0.34 | Cyanobacteria | 0.14 |
| Verrucomicrobia | 0.30 | Tenericutes | 0.10 |
| Class | Frequency (%) | Class | Frequency (%) |
| Clostridia | 45.39 | Bacteroidia | 39.20 |
| Bacteroidia | 17.11 | Fusobacteria | 24.72 |
| Fusobacteria | 17.05 | Clostridia | 21.69 |
| Erysipelotrichi | 8.07 | Gammaproteobacteria | 3.48 |
| Unclassified at Class level | 3.13 | Unclassified at Class level | 2.89 |
| Gammaproteobacteria | 2.96 | Betaproteobacteria | 2.41 |
| Bacilli | 1.78 | Erysipelotrichi | 2.35 |
| Betaproteobacteria | 1.14 | Flavobacteriia | 0.94 |
| Order | Frequency (%) | Order | Frequency (%) |
| Clostridiales | 41.56 | Bacteroidales | 39.20 |
| Bacteroidales | 17.11 | Fusobacteriales | 24.72 |
| Fusobacteriales | 17.05 | Clostridiales | 20.98 |
| Erysipelotrichales | 8.07 | Unclassified at Order level | 3.21 |
| Coriobacteriales | 3.70 | Aeromonadales | 3.01 |
| Unclassified at Order level | 3.49 | Burkholderiales | 2.36 |
| Aeromonadales | 2.51 | Erysipelotrichales | 2.35 |
| Burkholderiales | 1.13 | Flavobacteriales | 0.94 |
| Family | Frequency (%) | Family | Frequency (%) |
| Lachnospiraceae | 20.77 | Bacteroidaceae | 25.66 |
| Fusobacteriaceae | 17.02 | Fusobacteriaceae | 24.63 |
| Clostridiaceae | 8.91 | Lachnospiraceae | 9.24 |
| Bacteroidaceae | 6.89 | Prevotellaceae | 8.30 |
| Veillonellaceae | 5.29 | Unclassified at Family level | 4.94 |
| Unclassified at Family level | 5.14 | Clostridiaceae | 4.65 |
| Prevotellaceae | 4.85 | Veillonellaceae | 3.85 |
| Paraprevotellaceae | 4.69 | Paraprevotellaceae | 3.83 |
| Genus | Frequency (%) | Genus | Frequency (%) |
| Fusobacterium | 14.64 | Bacteroides | 25.66 |
| Blautia | 13.65 | Fusobacterium | 18.15 |
| Unclassified at Genus level | 7.13 | Prevotella | 11.64 |
| Bacteroides | 6.89 | Unclassified at Genus level | 9.69 |
| Clostridium | 6.65 | Blautia | 6.17 |
| Prevotella | 6.60 | Clostridium | 4.11 |
| Ruminococcus | 6.05 | Anaerobiospirillum | 2.81 |
| Megamonas | 3.81 | Megamonas | 2.55 |
| Species | Frequency (%) | Species | Frequency (%) |
| Unclassified at Species level | 37.59 | Unclassified at Species level | 32.41 |
| Fusobacterium gonidiaformans | 14.38 | Fusobacterium gonidiaformans | 17.73 |
| Clostridium hiranonis | 5.33 | Bacteroides denticanum | 9.46 |
| Blautia coccoides | 4.63 | Prevotella copri | 7.86 |
| Prevotella copri | 4.60 | Bacteroides rodentium | 3.90 |
| Paraprevotella clara | 2.94 | Anaerobiospirillum succiniciproducens | 2.75 |
| Collinsella intestinalis | 2.69 | Clostridium hiranonis | 2.22 |
| Bacteroides denticanum | 2.33 | Blautia coccoides | 2.06 |

TABLE 2.18
Top eight phyla, class, order, family, genus and species with estimates
of the relative frequencies (percentage of total hits) for dog 18
(Asher) before and after administration of EquiNectar ™.

| Before EquiNectar ™ | | After EquiNectar ™ | |
|---|---|---|---|
| Phylum | Frequency (%) | Phylum | Frequency (%) |
| Firmicutes | 70.57 | Firmicutes | 54.43 |
| Fusobacteria | 17.23 | Fusobacteria | 18.68 |
| Proteobacteria | 6.70 | Bacteroidetes | 15.89 |
| Bacteroidetes | 2.93 | Actinobacteria | 3.53 |
| Unclassified at Phylum level | 1.89 | Proteobacteria | 2.87 |
| Actinobacteria | 0.34 | Unclassified at Phylum level | 2.76 |
| Tenericutes | 0.11 | Spirochaetes | 0.58 |
| Spirochaetes | 0.05 | Verrucomicrobia | 0.36 |
| Class | Frequency (%) | Class | Frequency (%) |
| Clostridia | 49.55 | Clostridia | 34.33 |
| Erysipelotrichi | 19.19 | Fusobacteria | 18.68 |
| Fusobacteria | 17.23 | Bacteroidia | 14.41 |
| Gammaproteobacteria | 4.31 | Bacilli | 13.77 |
| Unclassified at Class level | 2.85 | Erysipelotrichi | 5.97 |
| Bacteroidia | 2.74 | Unclassified at Class level | 4.29 |
| Bacilli | 1.62 | Actinobacteria | 3.49 |
| Betaproteobacteria | 1.42 | Gammaproteobacteria | 0.89 |
| Order | Frequency (%) | Order | Frequency (%) |
| Clostridiales | 40.68 | Clostridiales | 26.03 |
| Erysipelotrichales | 19.19 | Fusobacteriales | 18.68 |
| Fusobacteriales | 17.23 | Bacteroidales | 14.41 |
| Coriobacteriales | 8.75 | Coriobacteriales | 8.17 |
| Aeromonadales | 3.48 | Turicibacterales | 7.02 |
| Unclassified at Order level | 3.15 | Lactobacillies | 6.20 |
| Bacteroidales | 2.74 | Erysipelotrichales | 5.97 |
| Burkholderiales | 1.39 | Unclassified at Order level | 4.64 |
| Family | Frequency (%) | Family | Frequency (%) |
| Coprobacillaceae | 17.42 | Fusobacteriaceae | 18.63 |
| Fusobacteriaceae | 17.21 | Lachnospiraceae | 13.96 |
| Lachnospiraceae | 16.40 | Coriobacteriaceae | 8.17 |
| Veillonellaceae | 11.59 | Turicibacteraceae | 7.02 |
| Coriobacteriaceae | 8.75 | Unclassified at Family level | 6.55 |
| Clostridiaceae | 7.27 | Bacteroidaceae | 6.31 |
| Unclassified at Family level | 4.29 | Prevotellaceae | 5.91 |
| Ruminococcaceae | 4.28 | Clostridiaceae | 5.16 |
| Genus | Frequency (%) | Genus | Frequency (%) |
| Catenibacterium | 16.62 | Fusobacterium | 16.18 |
| Fusobacterium | 15.07 | Unclassified at Genus level | 8.75 |
| Blautia | 11.29 | Blautia | 7.50 |
| Megamonas | 10.40 | Turicibacter | 7.02 |
| Collinsella | 8.46 | Bacteroides | 6.31 |
| Clostridium | 6.63 | Prevotella | 6.28 |
| Unclassified at Genus level | 6.55 | Collinsella | 6.14 |
| Ruminococcus | 4.77 | Ruminococcus | 5.99 |
| Species | Frequency (%) | Species | Frequency (%) |
| Unclassified at Species level | 48.27 | Unclassified at Species level | 36.80 |
| Fusobacterium gonidiaformans | 14.82 | Fusobacterium gonidiaformans | 15.76 |
| Collinsella intestinalis | 8.20 | Collinsella intestinalis | 5.79 |
| Clostridium hiranonis | 5.79 | Prevotella copri | 5.71 |
| Blautia coccoides | 3.41 | Clostridium hiranonis | 2.74 |
| Megamonas funiformis | 2.54 | Turicibacter sanguinis | 2.40 |
| Catenibacterium mitsuokai | 2.31 | Ruminococcus gnavus | 2.24 |
| Prevotella copri | 1.55 | Bifidobacterium saeculare | 2.21 |

According to Hullar et al. (supra), there are five predominant phyla in the canine gastrointestinal tract, namely Bacteroidetes, Firmicutes, Fusobacteria, Proteobacteria and Actinobacteria, the latter at lower levels. There are small quantitative variations between individual healthy dogs, probably reflecting diet and environmental factors as well as the microbiome established shortly after birth. It is generally agreed that the faecal microbiome is a good representation of the gastrointestinal tract microbiome so most work is done on faecal samples.

Gastrointestinal tract problems result in changes to the microbiome and to the metabolome, the metabolites produced by the gut bacteria. These include short-chain fatty acids (SCFAs) and products of amino acid metabolism. However, most work has been done on the changes to the microbiome. In disease states such as inflammation, losses in certain bacterial species leave ‘ecological niches’ which are rapidly colonised by other species. Currently, there is little consensus in the literature on which species are associated with specific disease states, although, according to Cassmann et al. (‘Alterations of the ileal and colonic mucosal microbiota in canine chronic enteropathies’, PLOS One, 11, 2, e0147321 (2016)), increased numbers of Enterobacteriaceae such as Escherichia coli, which are facultative anaerobes, are generally associated with CIBD. According to Minamoto et al. (supra), dogs with CIBD are known to have more Gammaproteobacteria and fewer Bacteroidia and, according to Xu et al. (‘Does canine inflammatory bowel disease influence gut microbial profile and host metabolism?’, BMC Vet. Res. 12, 1, 114 (2016)), dogs with CIBD also have less Lactobacillus. According to Hullar et al. (supra), dogs with GDV had a significant expansion of the Actinobacteria and again a much lower abundance of Bacteroidetes.

The various canine chronic enteropathies (CCE) are believed to be caused by an aberrant immune response towards this changed microbiome and several therapeutic approaches have therefore been tried. Bacterial populations in dogs can be altered by supplementation with, according to Pinna et al. (‘Influence of dietary protein and fructooligosaccharides on faecal fermentative end-products, faecal bacterial populations and apparent total tract digestability in dogs’, BMC Vet. Res., 14, 106 (2018)), fructooligosaccharides (FOS) as a prebiotics and, according to Strompfova et al. (‘Health benefits observed after probiotic Lactobacillus fermentum CCM 7421 application in dogs’, Appl. Microbiol. Biotechnol., 101, 16, 6309 (2017)), canine-derived probiotic strain Lactobacillus fermentum CCM 7421, although the benefit was small, and also, according to Herstad et al. (‘A diet change from dry food to beef induces reversible changes on the faecal microbiota in healthy, adult client-owned dogs’, BMC Vet. Res., 13, 1, 147 (2017)) and Pinna et al. (‘In vitro influence of dietary protein and fructooligosaccharides on metabolism of canine faecal microbiota’, BMC Vet. Res., 12, 53 (2016)), by high-protein diets which improved the SCFA profile but increased levels of toxic metabolites such as ammonia. The ideal supplement therefore would be one which optimised the existing canine microbiome rather than one which attempted to over-ride it.

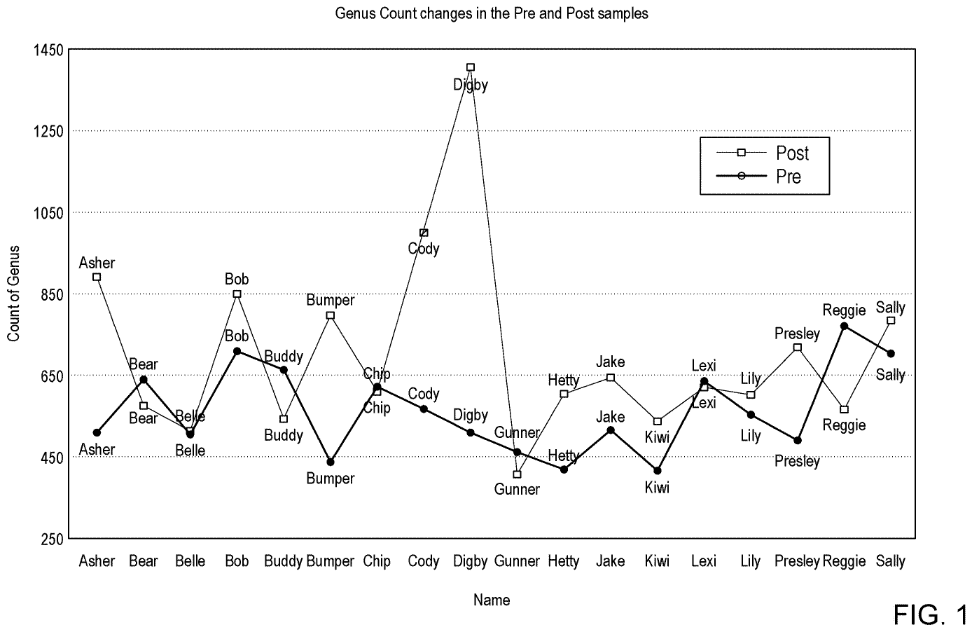

FIG. 1 is a graph of count of genus versus dog before and after administration of EquiNectar™ and shows that the diversity of gut bacteria generally increased following administration of EquiNectar™, which is reflective of improved gut and hence overall health even in dogs which were nominally healthy. Indeed it was observed that as a Class, Spirochaetes are down (which indicates a healthier gut microbiome) following administration of EquiNectar™ while as a Genus, Bacteroides, Prevotella, Ruminococcus and Blautia are all up (which also indicates a healthier gut microbiome) following administration of EquiNectar™.

Table 2.19 shows the relative frequencies (percentage of total hits) of Borrelia burgdorferi for the named dogs before (Pre) and after (Post) administration of EquiNectar™.

TABLE 2.19
The relative frequencies (percentage of total hits)
of Borrelia burgdorferi for the named dogs before (Pre)
and after (Post) administration of EquiNectar ™.

| | Number of | Number of | % Hits | % Hits |
|---|---|---|---|---|
| Dog Name | Hits Pre | hits Post | Pre | Post |
| Asher | 32 | 4 | 0.032 | 0.003 |
| Bear | 20 | 6 | 0.012 | 0.004 |
| Bob | 20 | 20 | 0.021 | 0.014 |
| Buddy | 58 | 10 | 0.061 | 0.011 |
| Bumper | 24 | 28 | 0.035 | 0.007 |
| Chip | 12 | 11 | 0.016 | 0.010 |
| Cody | 42 | 0 | 0.044 | 0 |
| Digby | 20 | 0 | 0.022 | 0 |
| Gunner | 2 | 0 | 0.003 | 0 |
| Hetty | 7 | 0 | 0.008 | 0 |
| Jake | 3 | 0 | 0.004 | 0 |
| Kiwi | 31 | 6 | 0.046 | 0.004 |
| Lily | 6 | 3 | 0.004 | 0.003 |
| Presley | 24 | 8 | 0.028 | 0.003 |
| Reggie | 50 | 7 | 0.041 | 0.005 |
| Sally | 22 | 7 | 0.024 | 0.004 |

In particular, it was noted that most dogs from Example 2 had lower levels of the bacterial species Borrelia burgdorferi after administering of EquiNectar™. This is the Spirochaete (class) that is responsible for Lyme disease. Dogs are known to suffer from Lyme disease, like humans, and are also carriers of the bacterium so that they can act as a host reservoir to infect humans and other animals. However, after treatment with Equinectar™ (the malt-based feed supplement containing ERME as previously described), Borrelia burgdorferi was substantially reduced or eliminated from the canine faecal samples.

### Example 3: Results of a Selected Ion-Flow Tube Mass Spectroscopy (SIFT-MS) Dog Study

Five adult dogs were fed 15 ml EquiNectar™ twice daily for four weeks as a spread over their normal dry diet and a sample of faeces was taken from each of five dogs at the start of the study and again after four weeks. The samples were stored frozen at −80° C. until the whole collection was transferred to the Open University (Milton Keynes) for volatile organic compounds (VOCs) analysis by selected ion-flow tube mass spectrometry (SIFT-MS). Faecal samples were taken from a −80° C. freezer and thoroughly defrosted. Exactly 5 g of each sample was weighed out and placed into a sample bag constructed from Nalophan tubing. The bag was filled with zero grade (hydrocarbon free) air before being sealed with a Swagelok fitting, then placed in an incubator at 40° C. for 45 minutes to increase compound volatilization. After incubation, the samples were then attached to the SIFT-MS via a heated sampling capillary. SIFT-MS is a real-time trace gas analyser where selected precursor ions (H₃O⁺, NO⁺ or O₂⁺ generated in an air/water mixture via a microwave discharge and then selected via an upstream quadrupole mass filter) are injected into helium carrier gas, and passed along a flow tube into which the sample is introduced via the heated sampling capillary. The precursor ions react with the sample VOCs and the resulting product ions are then separated in a downstream quadrupole mass filter before being detected and counted.

According to Fritsch et al. (‘Select dietary fibres alter the gastrointestinal microbiome composition and promote fermentative metabolism in the lower gastrointestinal tract of healthy adult dogs’, Current Developments in Nutrition, 3, Suppl. 1, (2019)), the canine microbiome is balanced between saccharolytic bacteria (SB) which metabolise fibre and complex carbohydrates and proteolytic bacteria (PB) which break down proteins. Intestinal health relies on both types being present but it is believed that an excess of the proteolytic bacteria (for example, Desulfovibrionaceae) is linked with gut dysfunction.

The various components of the microbiome produce volatile metabolites (VOCs) and measurement of these compounds gave information on the ratio of SB to PB. PB species have been linked with production of ammonia and alcohols and these chemicals (with others) were specifically measured by selected ion-flow tube mass spectroscopy (SIFT/MS). In all, the concentrations of ammonia, acetone, methanol, ethanol, propanol, acetaldehyde, acetic acid, propionic acid, butyric acid, hydrogen sulphide, methanethiol, dimethylsulfide, dimethyldisulfide, butanone, pentanone, toluene, and butanol in faecal samples were determined. The results are summarised in table 3.

TABLE 3
The concentration of faecal volatile metabolites
(ppb) (VOCs) before and after administration Equinectar ™.

| Metabolite | | | | | | | | | | |
|---|---|---|---|---|---|---|---|---|---|---|
| concentration | Dog 1 | | Dog 2 | | Dog 3 | | Dog 4 | | Dog 5 | |
| (ppb) | Before | After | Before | After | Before | After | Before | After | Before | After |
| Ammonia | 385 | 305 | 1340 | 970 | 1097 | 690 | 1902 | 1129 | 3127 | 543 |
| Acetone | 200 | 157 | 3067 | 9956 | 1240 | 2034 | 568 | 300 | 685 | 989 |
| Methanol | 12878 | 6473 | 9691 | 5368 | 23142 | 16969 | 10251 | 7447 | 7013 | 4907 |
| Ethanol | 27539 | 17796 | 19704 | 21542 | 4770 | 10088 | 2519 | 1805 | 411 | 7065 |
| Propanol | 8418 | 9029 | 3374 | 7351 | 5118 | 6179 | 3228 | 1396 | 545 | 2643 |
| Acetaldehyde | 259 | 356 | 109 | 438 | 221 | 533 | 165 | 96 | 35 | 287 |
| Acetic acid | 1092 | 646 | 624 | 636 | 325 | 1400 | 353 | 97 | 58 | 277 |
| Propionic acid | 817 | 632 | 801 | 584 | 481 | 2632 | 339 | 80 | 86 | 169 |
| Butyric acid | 372 | 261 | 226 | 406 | 102 | 482 | 48 | 102 | 108 | 150 |
| Hydrogen sulphide | 245 | 12 | 70 | 357 | 147 | 95 | 20 | 23 | 21 | 63 |
| Methanethiol | 129 | 149 | 251 | 208 | 25 | 92 | 4 | 181 | 3 | 147 |
| Dimethylsulphide | 35 | 42 | 80 | 71 | 63 | 102 | 103 | 50 | 0 | 25 |
| Dimethyldisulphide | 10 | 8 | 28 | 31 | 7 | 73 | 83 | 7 | 7 | 90 |
| Butanone | 23 | 18 | 29 | 318 | 48 | 219 | 78 | 49 | 51 | 0 |
| Pentanone | 10 | 5 | 29 | 20 | 0 | 55 | 23 | 5 | 15 | 14 |
| Toluene | 61 | 6 | 73 | 109 | 5 | 7 | 0 | 6 | 0 | 11 |
| Butanol | 765 | 495 | 1107 | 695 | 479 | 1260 | 433 | 564 | 821 | 815 |

The results showed that all the dogs, which were healthy and without reported problems, had relatively low levels of metabolites from more toxic bacteria (PB). Nevertheless, all dogs showed a reduction in at least one of these compounds (ammonia, methanol, ethanol, hydrogen sulphide, toluene, butanol, methanethiol, dimethylsulfide) while 4 out of 5 also showed an increase in beneficial metabolites generated by SB species (acetone, the short chain fatty acids acetic acid, butyric acid, propionic acid).

In conclusion, even in healthy dogs, Equinectar™ alters the gut microbiome in dogs to improve the ratio of favourable to unfavourable species of bacteria.

### Example 4: A Combined Microbiome and Metabolomics Study

Dogs (9) (6 aged 2-5 and 3 aged 5+ years old and numbered in the Figures hereinbelow) were recruited from a government organisation (UK Border Force). They were all working dogs on a similar diet and living in similar environments. The dogs were fed Equinectar™ (0.5 ml/kg body weight twice daily) as a spread over their normal dry diet for 4 weeks and a sample of faeces was taken from each dog at the start of the study and again after four weeks. The samples were stored frozen at −80° C. until the gut microbiome content was determined by 16S metagenomic analysis (Illumina Incorporated (CA, US) platform) and the gut microbiome profiled using error-corrected 454 pyrosequencing data from the 16S rRNA amplicons according to Example 2 and analysis for volatile organic compounds (VOCs) by selected ion-flow tube mass spectrometry (SIFT-MS) as described in Example 3.

The dog handlers also noted changes in the appetite, behaviour, body, coat, droppings and focus of their dogs.

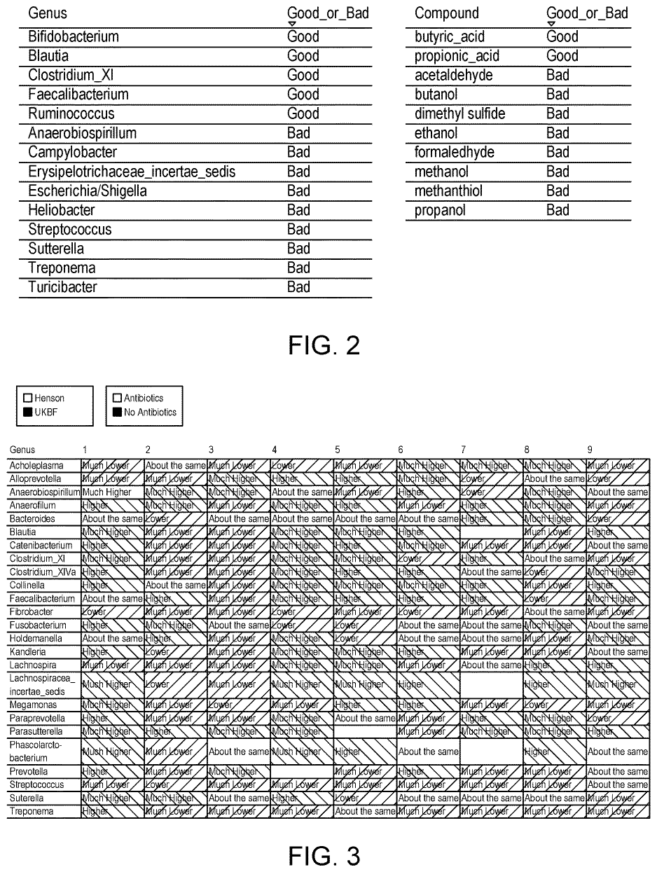

FIG. 2 shows a list of bacterial genera and faecal volatile metabolites (VOCs) associated with ‘good’ and ‘bad’ health.

FIG. 3 shows the top 25 abundant bacterial genera changes per dog (all not on antibiotics) after administration of Equinectar™. Plus or minus 20%=About the Same, 20 to 50% lower=Lower, 50 to 100% Lower=Much Lower, exists Pre but not Post=Lost, 20 to 100% higher=Higher, 100+% higher=Much Higher, and doesn't exist Pre but does Post=Gained.

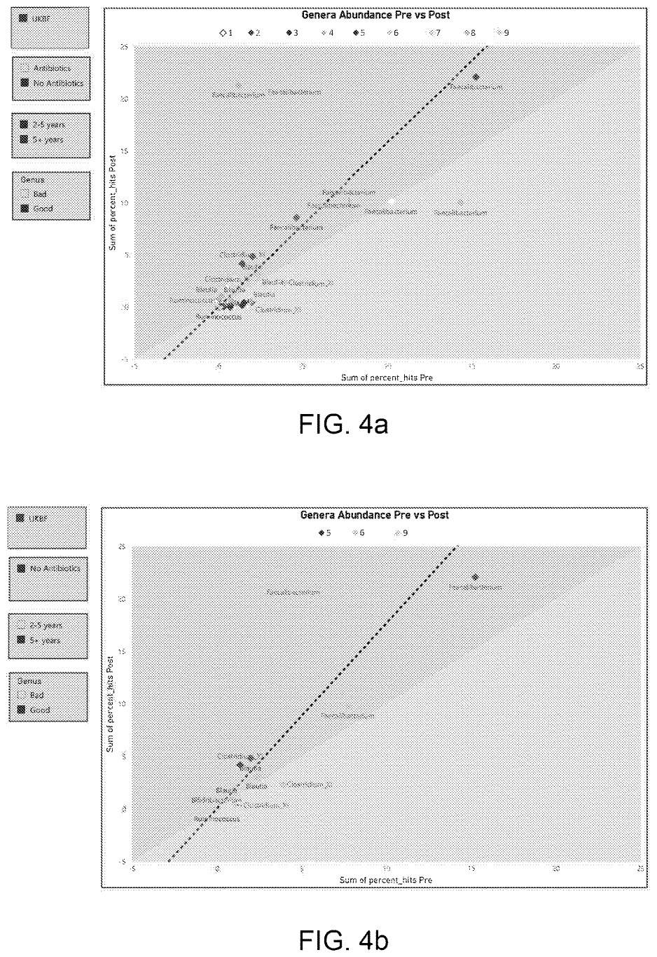

FIGS. 4a and 4b show scatter plots comparing pre- (x axis) and post-(y axis) administration of Equinectar™ for ‘good’ bacterial genera relative abundance (via percent hits) per dog (all not on antibiotics) and only those aged at least 5 years (not on antibiotics) respectively. The dotted line shows that the ‘good’ bacteria are increasingly abundant post treatment with Equinectar™. This is even clearer in dogs aged at least 5 years.

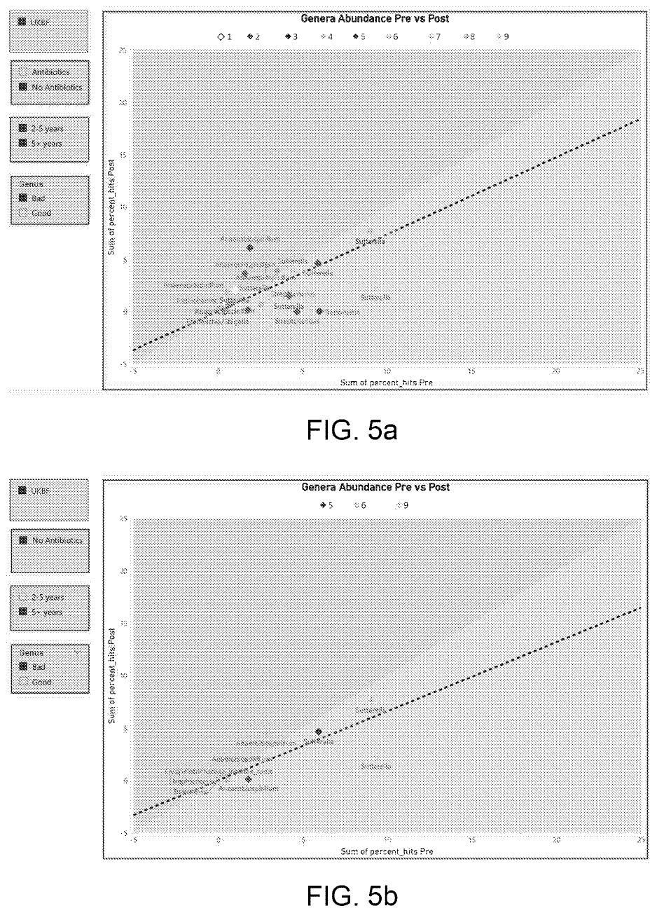

FIGS. 5a and 5b show scatter plots comparing pre- (x axis) and post-(y axis) administration of Equinectar™ for ‘bad’ bacterial genera relative abundance (via percent hits) per dog (all not on antibiotics) and only those aged at least 5 years (not on antibiotics) respectively. The dotted line shows that the ‘bad’ bacteria are increasingly less abundant post treatment with Equinectar™. This is even clearer in dogs aged at least 5 years.

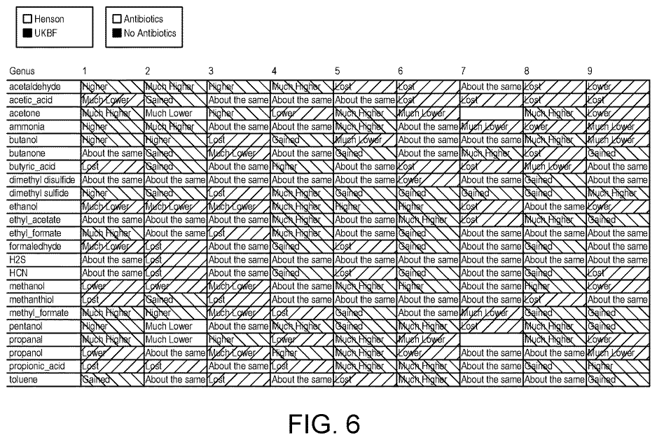

FIG. 6 shows the volatile organic compound (VOC) changes per dog (all not on antibiotics) after administration of Equinectar™. Plus or minus 20%=About the Same, 20 to 50% lower=Lower, 50 to 100% Lower=Much Lower, exists Pre but not Post=Lost, 20 to 100% higher=Higher, 100+% higher=Much Higher, and doesn't exist Pre but does Post=Gained.

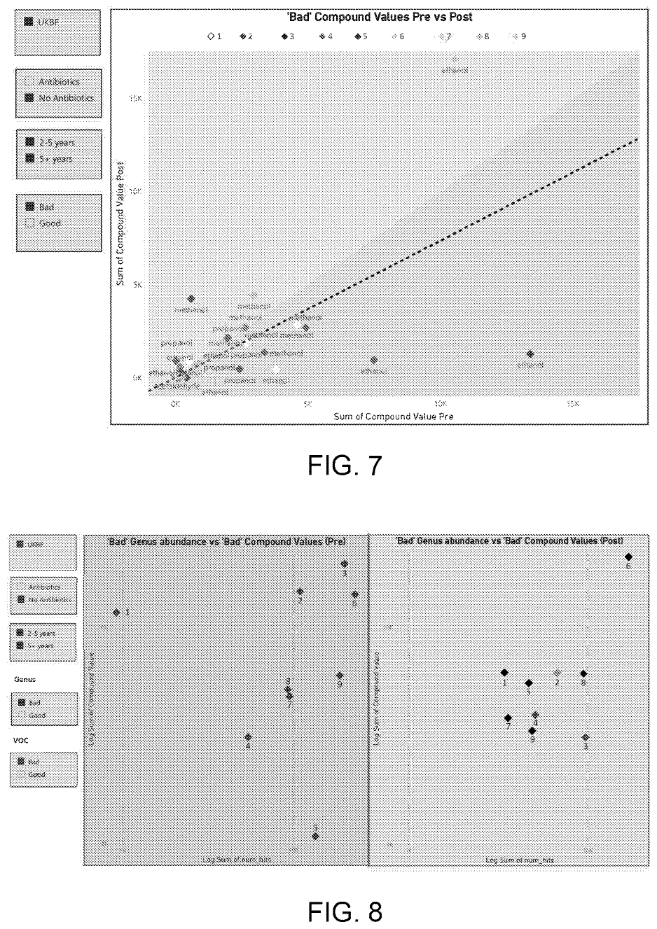

FIG. 7 shows a scatter plot comparing pre- (x axis) and post-(y axis) administration of Equinectar™ for ‘bad’ VOCs (VOCs related to poor health) relative abundance (via percent hits) per dog (all not on antibiotics). The dotted line shows that the ‘bad bacteria are increasingly less abundant post treatment with Equinectar™.

FIG. 8 shows ‘bad’ VOCs versus ‘bad’ bacterial genera per dog (all dogs not on antibiotics) pre- and post-administration of Equinectar™. After administration of Equinectar™ the levels of both ‘bad’ VOCs and ‘bad’ bacterial genera generally decrease.

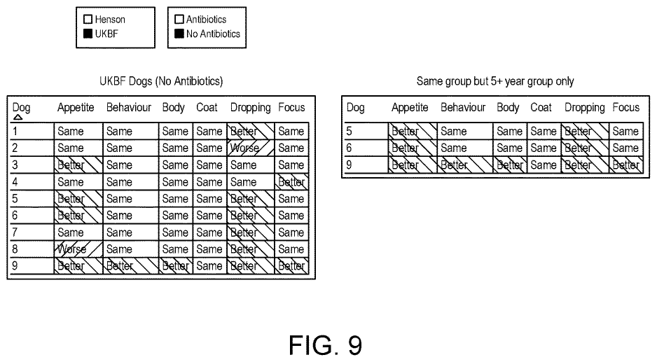

FIG. 9 shows changes in the appetite, behaviour, body, coat, droppings and focus of the dogs (not on antibiotics). For droppings, ideally the dog handler should be able to pick up the droppings and they should neither be rock hard nor sloppy in consistency. While most of the dogs had very little room for improvement which resulted in the vast majority of ratings to be categorised as ‘Same’, there was a notable improvement in dropping quality for more than half the selected dogs and appetite improvements were also seen in just under half the dogs.

As can be seen, Equinectar™ greatly improved the levels of ‘good’ bacteria (Blautia, Faecalibacterium, Ruminococcus) in the dogs and reduced the amount of pathogenic bacteria (Proteobacteria, especially Anaerobiospirillum, Campylobacter, Erysipelotrichaceae incertae sedis, Escherichia/Shigella, Helicobacter, Sutterella), Spirochaetes, Streptococcus and Treponema. Overall, although the picture was complex, all dogs either had higher levels of ‘good’ VOCs (butyric acid, ethyl acetate, propionic acid) or reduced levels of toxic metabolites (ammonia, butanol, ethanol, formaldehyde, methanol, methanethiol).

These dogs were all in excellent condition so would not be expected to derive major benefit from Equinectar™. Nevertheless, analysis of the results showed that generally there was improved appetite and quality of droppings. This was particularly found for older dogs (>5 years) and reflects an improvement in the microbiome (reduced levels of Treponema and Propiogenium). As dogs age, they have more gastrointestinal issues so Equinectar™ would be expected to be more beneficial for the older animal. The increase in canine gut health with a more solid dropping means a better experience for the dogs' handlers.

Example 5: Metabolomic Results from Dogs Living in a Domestic Environment

These dogs (4) were chosen to represent ordinary domestic dogs (2 Labradors, whippet, Yorkshire terrier) and were fed Equinectar™ as above in Example 4.

Thus, the dogs were fed Equinectar™ (0.5 ml/kg body weight twice daily) as a spread over their normal dry diet for 4 weeks and a sample of faeces was taken from each dog at the start of the study and again after four weeks. The samples were stored frozen at −80° C. and analysed for volatile organic compounds (VOCs) by selected ion-flow tube mass spectrometry (SIFT-MS) as described in Example 3.

The results are summarised in Table 4.

TABLE 4
The concentration of faecal volatile metabolites (ppb) before
(22 Jun. 2023) and after (20 Jul. 2023 administration Equinectar ™.

| Metabolite | | | | | | | | |
|---|---|---|---|---|---|---|---|---|
| concentration | Dog 1 | | Dog 2 | | Dog 3 | | Dog 4 | |
| (ppbv) | Before | After | Before | After | Before | After | Before | After |
| Ammonia | 232 | 255 | 339 | 278 | 468 | 338 | 111 | 138 |
| Acetone | 408 | 1668 | 447 | 300 | 685 | 84 | 467 | 20 |
| Methanol | 2924 | 4432 | 5223 | 8641 | 6031 | 6709 | 6428 | 8382 |
| Ethanol | 61033 | 50307 | 45185 | 27866 | 11338 | 16765 | 22905 | 18307 |
| Propanol | 12808 | 9661 | 4457 | 6753 | 2524 | 1790 | 4460 | 2975 |
| Acetaldehyde | 107 | 304 | 416 | 278 | 282 | 887 | 222 | 172 |
| Acetic acid | 2068 | 1154 | 477 | 1621 | 1058 | 1547 | 312 | 462 |
| Propionic acid | 3522 | 2141 | 902 | 5614 | 4174 | 6157 | 78 | 2007 |
| Butyric acid | 1536 | 1016 | 529 | 858 | 639 | 800 | 212 | 287 |
| Hydrogen sulphide | 58 | 62 | 117 | 78 | 174 | 104 | 39 | 29 |
| Methanethiol | 268 | 223 | 110 | 116 | 95 | 44 | 56 | 46 |
| Dimethylsulphide | 49 | 82 | 219 | 221 | 134 | 202 | 93 | 151 |
| Dimethyldisulphide | 648 | 385 | 1039 | 315 | 237 | 123 | 156 | 113 |
| Butanone | 45 | 202 | 12 | 6 | 54 | 6 | 28 | 25 |
| Pentanol | 2231 | 1340 | 539 | 957 | 696 | 764 | 469 | 226 |
| Toluene | 323 | 220 | 158 | 484 | 405 | 494 | 6 | 15 |
| Butanol | 6941 | 6544 | 3205 | 2669 | 5580 | 3966 | 825 | 959 |
| Methyl formate | 572 | 357 | 116 | 462 | 306 | 415 | 94 | 138 |
| Ethyl formate | 2163 | 1333 | 561 | 3710 | 2420 | 3970 | 7 | 1394 |
| Ethyl acetate | 1011 | 689 | 314 | 601 | 339 | 479 | 166 | 189 |
| Indole | 146 | 54 | 18 | 17 | 17 | 17 | 0 | 0 |

These dogs had a different environment and diet from the dogs in Example 4. However, the VOC findings were very similar, with generally raised levels of ethyl acetate, butyric acid and propionic acid and reduced levels of toxins such as hydrogen sulphide, methanethiol, butanol, ethanol and propanol, showing an improved metabolomic response.

1. A feed additive for a dog, the feed additive comprising:
a malt extract, the malt extract comprising one or more enzymes selected from the group consisting of amylases, maltases, cellulases, fructanases, glucanases, xylanases and deacetylases.

2. A method of reducing diarrhoea in a dog, the method comprising providing a therapeutically effective amount of the feed additive of claim 1 to the dog for ingestion, wherein the dog has a weight of 20-200 kg.

3. A method of decreasing the level of a harmful digestive metabolite or increasing the level of a beneficial digestive metabolite in a dog gut, the method comprising providing a therapeutically effective amount of the feed additive of claim 1 to the dog for ingestion, wherein the harmful digestive metabolite is selected from the group consisting of ammonia, methanol, ethanol, hydrogen sulphide, toluene, butanol, methanethiol, and dimethylsulfide, and wherein the beneficial digestive metabolite is selected from the group consisting of acetone, acetic acid, butyric acid, and propionic acid.

4. A method of increasing the food appetite and/or diversity of gut bacteria in a dog, the method comprising providing a therapeutically effective amount of the feed additive of claim 1 to the dog for ingestion.

5. A method of increasing the amount of bacterial genus Faecalbacterium in a dog digestive system, the method comprising providing a therapeutically effective amount of the feed additive of claim 1 to the dog for ingestion.

6. A method of decreasing the amount of bacterial genera Streptococcus and/or Treponema in a dog digestive system, the method comprising providing a therapeutically effective amount of the feed additive of claim 1 to the dog for ingestion.

7. A method of preventing or treating Lyme disease in a dog, the method comprising providing a therapeutically effective amount of the feed additive of claim 1 to the dog for ingestion.

8. A method of reducing or preventing the spread of Lyme disease by dogs, the method comprising providing a therapeutically effective amount of the feed additive of claim 1 to the dog for ingestion.

9. The feed additive according to claim 1, wherein the malt extract additionally comprises one or more proteinases and/or lipases.

10. The feed additive according to claim 1, wherein the malt extract additionally comprises one or more water soluble sugars selected from the group consisting of maltose, maltotriose, and maltose polymers.

11. The feed additive according to claim 1, wherein the malt extract is based on one of the seeds selected from the group consisting of barley, wheat, triticale, sorghum, maize, buck wheat, rice and a mixture thereof.

12. The feed additive according to claim 1, wherein a daily dosage of the malt extract is 0.3-30 g per 1000 g dog body weight.

13. The feed additive for use according to claim 1, wherein the diastatic power of the malt extract is above 35 degrees Lintner.

14. The feed additive according to claim 1, wherein the feed additive additionally comprises one or more medium chain triglycerides.

15. The feed additive according to claim 14, wherein the feed additive comprises 1-15% w/w one or more medium chain triglycerides.

16. The feed additive according to claim 14, wherein the medium chain triglyceride is coconut oil.

17. The feed additive according to claim 1, wherein the feed additive additionally comprises an effective amount of one or more water soluble organic acid or salt thereof.

18. The feed additive according to claim 17, wherein the water soluble organic acid is selected from the group consisting of caprylic acid, sorbic acid, caproic acid, benzoic acid, ascorbic acid, propionic acid, acetic acid, formic acid, fumaric acid, and tartaric acid.

19. The feed additive according to claim 17, wherein the feed additive comprises 0.001-5 w/w of one or more water soluble organic acid or salt thereof.

20. A dog feed comprising the feed additive according to claim 1.
